SEQUENCE LISTING (54 sequences)

SEQ ID NO: 1 - RNA, 23 nt, Artificial Sequence
  Description of Artificial Sequence Synthetic oligonucleotide
gggagacaagaauaaacgcucaa

SEQ ID NO: 2 - RNA, 24 nt, Artificial Sequence
  Description of Artificial Sequence Synthetic oligonucleotide
aacgacaggaggcucacaacagga

SEQ ID NO: 3 - RNA, 50 nt, Artificial Sequence
  Description of Artificial Sequence Synthetic oligonucleotide
gcgcgggaauaguauggaaggauacguauaccgugcaauccagggcaacg

SEQ ID NO: 4 - RNA, 50 nt, Artificial Sequence
  Description of Artificial Sequence Synthetic oligonucleotide
gggcuucauaagcuacaccggccaacgcagaaaugccuuaagcccgaguu

SEQ ID NO: 5 - RNA, 50 nt, Artificial Sequence
  Description of Artificial Sequence Synthetic oligonucleotide
ggccauagcgcaccaccaagagcaaaucccuaagcgcgacucgagugagc

SEQ ID NO: 6 - RNA, 50 nt, Artificial Sequence
  Description of Artificial Sequence Synthetic oligonucleotide
gggccaaucgaagccgguaauucccaaacuaacgugcaaacugcacccgc

SEQ ID NO: 7 - RNA, 49 nt, Artificial Sequence
  Description of Artificial Sequence Synthetic oligonucleotide
gcgguauguagggaauagcacuuuuuuugcguauaccuacaccgcagcg

SEQ ID NO: 8 - RNA, 50 nt, Artificial Sequence
  Description of Artificial Sequence Synthetic oligonucleotide
aggcgagcccgaccacgucaguaugcuagacaacaacgcccgcgugguac

SEQ ID NO: 9 - RNA, 51 nt, Artificial Sequence
  Description of Artificial Sequence Synthetic oligonucleotide
ccccgcuuuuugacgugaucgaacgcguaucaguaacgucagcagucgagc

SEQ ID NO: 10 - RNA, 51 nt, Artificial Sequence
  Description of Artificial Sequence Synthetic oligonucleotide
caaagcguguauucucgugagccgaccaucguugcgaacauccccggaacg

SEQ ID NO: 11 - RNA, 48 nt, Artificial Sequence
  Description of Artificial Sequence Synthetic oligonucleotide
gacccguaugaagguggcgcaggacacgaccgucugcaaugagcgagc

SEQ ID NO: 12 - RNA, 50 nt, Artificial Sequence
  Description of Artificial Sequence Synthetic oligonucleotide
ccgaccuguacagcaguuaguuacacguuugaaacaaccggcguucgagc

SEQ ID NO: 13 - RNA, 50 nt, Artificial Sequence
  Description of Artificial Sequence Synthetic oligonucleotide
ggcuuacacggagaaacaagagagcggcccaaacuugauugacaguggcc

SEQ ID NO: 14 - RNA, 49 nt, Artificial Sequence
  Description of Artificial Sequence Synthetic oligonucleotide
ggcccuuaacgcaaaaacgaaggaucaucgauugaucgccuuaugggcu

SEQ ID NO: 15 - RNA, 48 nt, Artificial Sequence
  Description of Artificial Sequence Synthetic oligonucleotide
ccgcggucugugggacccuucaggaugaagcggcaacccaugcgggcc

SEQ ID NO: 16 - DNA, 40 bp, Artificial Sequence
  Description of Artificial Sequence Synthetic primer
taatacgactcactatagggagacaagaataaacgctcaa

SEQ ID NO: 17 - DNA, 24 bp, Artificial Sequence
  Description of Artificial Sequence Synthetic primer
tcctgttgtgagcctcctgtcgtt

SEQ ID NO: 18 - RNA, 24 nt, Artificial Sequence
  Description of Artificial Sequence Synthetic oligonucleotide
gggagacaagaauaaacgcucaag

SEQ ID NO: 19 - RNA, 30 nt, Artificial Sequence
  Description of Artificial Sequence Synthetic oligonucleotide
gggagacaagaauaaacgcucaagcgguau

SEQ ID NO: 20 - RNA, 39 nt, Artificial Sequence
  Description of Artificial Sequence Synthetic oligonucleotide
caauccagggcaacgaacgacaggaggcucacaacagga

SEQ ID NO: 21 - RNA, 33 nt, Artificial Sequence
  Description of Artificial Sequence Synthetic oligonucleotide
accgcagcgaacgacaggaggcucacaacagga

SEQ ID NO: 22 - RNA, 97 nt, Artificial Sequence
  Description of Artificial Sequence Synthetic oligonucleotide
gggagacaagaauaaacgcucaagcgcgggaauaguauggaaggauacguauaccgugcaauccagggcaacgaacgacaggaggcucacaacagga

SEQ ID NO: 23 - RNA, 34 nt, Artificial Sequence
  Description of Artificial Sequence Synthetic oligonucleotide
cgcgggaauaguauggaaggauacguauaccgug

SEQ ID NO: 24 - RNA, 24 nt, Artificial Sequence
  Description of Artificial Sequence Synthetic oligonucleotide
guagggaauagcacguauaccuac

SEQ ID NO: 25 - RNA, 96 nt, Artificial Sequence
  Description of Artificial Sequence Synthetic oligonucleotide
gggagacaagaauaaacgcucaagcgguauguagggaauagcacuuuuuuugcguauaccuacaccgcagcgaacgacaggaggcucacaacagga

SEQ ID NO: 26 - RNA, 7 nt, Artificial Sequence
  Description of Artificial Sequence Synthetic oligonucleotide
gcgguau

SEQ ID NO: 27 - RNA, 15 nt, Artificial Sequence
  Description of Artificial Sequence Synthetic oligonucleotide
caauccagggcaacg

SEQ ID NO: 28 - RNA, 9 nt, Artificial Sequence
  Description of Artificial Sequence Synthetic oligonucleotide
accgcagcg

SEQ ID NO: 29 - RNA, 8 nt, Artificial Sequence
  Description of Artificial Sequence Synthetic oligonucleotide
uggaagga

SEQ ID NO: 30 - RNA, 7 nt, Artificial Sequence
  Description of Artificial Sequence Synthetic oligonucleotide
cuuuuuu

SEQ ID NO: 31 - RNA, 97 nt, Artificial Sequence
  Description of Artificial Sequence Synthetic oligonucleotide
gggagacaagaauaaacgcucaaaggcgagcccgaccacgucaguaugcuagacaacaacgcccgcgugguacaacgacaggaggcucacaacagga

SEQ ID NO: 32 - RNA, 97 nt, Artificial Sequence
  Description of Artificial Sequence Synthetic oligonucleotide
gggagacaagaauaaacgcucaagggcuucauaagcuacaccggccaacgcagaaaugccuuaagcccgaguuaacgacaggaggcucacaacagga

SEQ ID NO: 33 - RNA, 96 nt, Artificial Sequence
  Description of Artificial Sequence Synthetic oligonucleotide
gggagacaagaauaaacgcucaaggcccuuaacgcaaaaacgaaggaucaucgauugaucgccuuaugggcuaacgacaggaggcucacaacagga

SEQ ID NO: 34 - RNA, 29 nt, Artificial Sequence
  Description of Artificial Sequence Synthetic oligonucleotide
nnnnnggaauagnnnnncguauacnnnnn

SEQ ID NO: 35 - RNA, 26 nt, Artificial Sequence
  Description of Artificial Sequence Synthetic oligonucleotide
nnnnggaauagnnnncguauacnnnn

SEQ ID NO: 36 - DNA, 97 bp, Artificial Sequence
  Description of Artificial Sequence Synthetic oligonucleotide
tcctgttgtgagcctcctgtcgttnnnnnnnnnnnnnnnnnnnnnnnnnnnnnnnnnnnnnnnnnnnnnnnnnnttgagcgtttattcttgtctccc

SEQ ID NO: 37 - DNA, 114 bp, Artificial Sequence
  Description of Artificial Sequence Synthetic oligonucleotide
tcctgttgtgagcctcctgtcgttnnnnnnnnnnnnnnnnnnnnnnnnnnnnnnnnnnnnnnnnnnnnnnnnnnttgagcgtttattcttgtctccctatagtgagtcgtatta

SEQ ID NO: 38 - DNA, 114 bp, Artificial Sequence
  Description of Artificial Sequence Synthetic oligonucleotide
taatacgactcactatagggagacaagaataaacgctcaannnnnnnnnnnnnnnnnnnnnnnnnnnnnnnnnnnnnnnnnnnnnnnnnnaacgacaggaggctcacaacagga

SEQ ID NO: 39 - DNA, 23 bp, Artificial Sequence
  Description of Artificial Sequence Synthetic oligonucleotide
ttgagcgtttattcttgtctccc

SEQ ID NO: 40 - DNA, 24 bp, Artificial Sequence
  Description of Artificial Sequence Synthetic oligonucleotide
aacgacaggaggctcacaacagga

SEQ ID NO: 41 - DNA, 40 bp, Artificial Sequence
  Description of Artificial Sequence Synthetic oligonucleotide
ttgagcgtttattcttgtctccctatagtgagtcgtatta

SEQ ID NO: 42 - DNA, 23 bp, Artificial Sequence
  Description of Artificial Sequence Synthetic oligonucleotide
gggagacaagaataaacgctcaa

SEQ ID NO: 43 - DNA, 40 bp, Artificial Sequence
  Description of DNA/RNA Molecule Synthetic oligonucleotide
taatacgactcactatagggagacaagaauaaacgcucaa

SEQ ID NO: 44 - RNA, 24 nt, Artificial Sequence
  Description of Artificial Sequence Synthetic oligonucleotide
aacgacaggaggcucacaacagga

SEQ ID NO: 45 - DNA, 50 bp, Artificial Sequence
  Description of Artificial Sequence Synthetic oligonucleotide
ggccatagcgcaccaccaagagcaaatccctaagcgcgactcgagtgagc

SEQ ID NO: 46 - DNA, 50 bp, Artificial Sequence
  Description of Artificial Sequence Synthetic oligonucleotide
aggcgagcccgaccacgtcagtatgctagacaacaacgcccgcgtggtac

SEQ ID NO: 47 - DNA, 50 bp, Artificial Sequence
  Description of Artificial Sequence Synthetic oligonucleotide
gggccaatcgaagccggtaattcccaaactaacgtgcaaactgcacccgc

SEQ ID NO: 48 - DNA, 50 bp, Artificial Sequence
  Description of Artificial Sequence Synthetic oligonucleotide
ggcttacacggagaaacaagagagcggcccaaacttgattgacagtggcc

SEQ ID NO: 49 - DNA, 50 bp, Artificial Sequence
  Description of Artificial Sequence Synthetic oligonucleotide
ccgacctgtacagcagttagttacacgtttgaaacaaccggcgttcgagc

SEQ ID NO: 50 - DNA, 51 bp, Artificial Sequence
  Description of Artificial Sequence Synthetic oligonucleotide
ccccgctttttgacgtgatcgaacgcgtatcagtaacgtcagcagtcgagc

SEQ ID NO: 51 - DNA, 51 bp, Artificial Sequence
  Description of Artificial Sequence Synthetic oligonucleotide
caaagcgtgtattctcgtgagccgaccatcgttgcgaacatccccggaacg

SEQ ID NO: 52 - DNA, 48 bp, Artificial Sequence
  Description of Artificial Sequence Synthetic oligonucleotide
ccgcggtctgtgggacccttcaggatgaagcggcaacccatgcgggcc

SEQ ID NO: 53 - DNA, 49 bp, Artificial Sequence
  Description of Artificial Sequence Synthetic oligonucleotide
gcggtatgtagggaatagcactttttttgcgtatacctacaccgcagcg

SEQ ID NO: 54 - DNA, 50 bp, Artificial Sequence
  Description of Artificial Sequence Synthetic oligonucleotide
gcgcgggaatagtatggaaggatacgtataccgtgcaatccagggcaacg

(19) United States
(12) Patent Application Publication
(10) Pub. No.: US 2008/0227735 A1
(43) Pub. Date: Sep. 18, 2008

(54) Aptamers Selected From Live Tumor Cells and the Use Thereof

(75) Inventors: Bertrand Tavitian, Paris (FR); Frederic Duconge, Sceaux (FR); Domenico Libri, Gif-sur-Yvette (FR); Vittorio De Franciscis, Naples (IT); Laura Cerchia, Naples (IT)

Correspondence Address: OBLON, SPIVAK, MCCLELLAND MAIER & NEUSTADT, P.C., 1940 DUKE STREET, ALEXANDRIA, VA, 22314, US

(73) Assignee: COMMISSARIAT A L'ENERGIE ATOMIQUE, PARIS FRANCE (FR)

(21) Appl. No.: 10/593,256

(22) PCT Filed: Mar. 17, 2005
(86) PCT No.: PCT/FR05/00656

§ 371 Date: Feb. 28, 2008

(30) Foreign Application Priority Data
Mar. 17, 2004 (FR) 0402774

(51) Int. Cl. A61K 31/7052; C40B 30/04; C12Q 1/68; A61P 35/00; C07H 21/00

(52) U.S. Cl. 514/44; 506/9; 536/23.1; 435/6

(57) ABSTRACT

The invention relates to aptamers selected from live tumor cells and to the use thereof for diagnosis and treatment of certain cancers and other pathologies.

The present invention relates to aptamers selected from live tumor cells and to uses thereof in the diagnosis and treatment of certain cancers and other pathologies.

In the oncology field, non-invasive diagnostic methods by in vivo imaging (radiography, X-ray scanner, MRI, gamma-scintigraphy, positron emission tomography), are rarely specific for a molecular determinant (or marker) characteristic of the tumor to be diagnosed or to be treated. This contrasts with the precision of the knowledge obtained in vitro, which describes increasingly finely the molecular anomalies which cause cancerous processes, and results in the diagnostic tools being inappropriate for the current data of molecular science. The same dichotomy between in vitro and in vivo is often found in the field of anticancer therapies, which results in difficulties in developing treatments with an acceptable therapeutic index (effective dose/toxic dose).

The search for ligands capable of recognizing a molecular determinant (or marker) signaling a specific type of tumor, or else a given stage in its development, or alternatively signaling the metabolic state of a tumor, is therefore essential for better follow-up and better therapy of cancers. Unfortunately, these ligands are generally obtained from targets which have been purified and isolated out of their biological context, and which are therefore different from the targets placed in their natural environment.

Thus, most commonly, the ligands, which are effective in a test tube, are incapable of interacting with their target:
because they cannot cross tissue barriers,
because they are instable in the organism, or responsible for too many adverse interactions with other biomolecules,
because the natural structure of the target, placed out of its natural cell environment, was not conserved or because certain essential modifications of this structure, such as: (i) post-translational modifications of proteins or (ii) interactions with other proteins, cannot be reproduced in vitro.

The latter two limitations are particularly frequent in the case of targets such as transmembrane proteins comprising a lipophilic segment inserted into the lipid cell membrane; this lipophilic segment is not conserved in vitro, whereas the membrane insertion of these proteins determines their structure and is essential to their activity.

Furthermore, the problem arises of the specificity with which the available ligands recognize the targets identified in tumors. It is therefore important to be able to provide specific ligands for diagnosing and/or treating certain cancers, in particular those related to the presence of a mutated tyrosine kinase receptor, resulting in constitutive activation or in over-expression of this receptor.

Molecular medicine therefore needs new molecular recognition probes which are:
specific,
adjustable, and
easy to produce at a reasonable cost.

Pharmacological research has set up novel strategies for discovering novel ligands effective against targets identified in tumors:
combinatorial libraries of small molecules make it possible to increase the chances of finding a ligand against a specific protein (example: a subclass of combinatorial libraries, false substrates which inhibit enzymes, such as those which inhibit MMPs (matrix metalloproteases)). Their major drawback is that they are sorted in vitro. Moreover, their selectivity is not guaranteed, hence the difficulty in obtaining, with these agents, a compound which is sufficiently selective to be effective and free of side effects. Only exceptionally are compounds obtained which are specific for an abnormal protein form, which leads to non-specific binding and adverse effects and results in a poor therapeutic index;
monoclonal antibodies are excellent agents for specific targeting and have recently been used for therapeutic purposes (example: Herceptin in breast cancer); however, they remain very difficult to use in vivo due to their sizes, idiotypy and immunogenicity, and are extremely expensive to produce and to optimize. Furthermore, monoclonal antibodies reach their limits when the recognition of a point mutation affecting a single amino acid on a protein is involved. In particular, no monoclonal antibody exists which is capable of identifying one of the abnormal forms of the Ret protein;
aptamers, which constitute an alternative means of diagnosis and therapy, and which have a certain number of advantages compared with antibodies, as illustrated in Table I below.

| | |
|---|---|
| Aptamers | Antibodies |

| Size | 9-15 | kDa | >150 | kDa |
|---|---|---|---|---|
| discrimination* | +++ | | +/++ | |
| affinity | 5-100 | nM | 0.1-100 | nM |

| | | |
|---|---|---|
| Source | synthetic | animal |
| Cost | [symbol] | [symbol] |

*theophylline versus caffeine, for example.

A method of selecting aptamers, which bind specifically and have a high affinity for predefined targets, was described at the beginning of the 1990s, and known as the SELEX method (Systematic Evolution of Ligands by Exponential enrichment). This method operates in iterative cycles of selection-amplification; this method and a certain number of improvements and applications of this method are described in particular in the following American patents: U.S. Pat. No. 5,270,163; U.S. Pat. No. 5,475,096; U.S. Pat. No. 5,496,938; U.S. Pat. No. 5,567,588; U.S. Pat. No. 5,580,737; U.S. Pat. No. 5,637,459; U.S. Pat. No. 5,660,985; U.S. Pat. No. 5,683,867; U.S. Pat. No. 5,707,796; U.S. Pat. No. 5,763,177 and U.S. Pat. No. 5,789,157.

Briefly, the principle of the SELEX method involves the selection, from a mixture of nucleic acids comprising random sequences, and by successive reiterations of binding, separation and amplification steps, of nucleic acid molecules (aptamers) exhibiting a defined binding affinity and a defined specificity for a given target. Thus, starting from a mixture of random nucleic acids, the SELEX method comprises more specifically the following steps:
bringing the mixture of nucleic acids into contact with the target element (natural or synthetic polymers: proteins, polysaccharides, glycoproteins, hormones, receptors, antigens, antibodies, cell surfaces; small molecules: medicament, metabolites, cofactors, substrates, transition state analogs; tissues and in particular whole cells; viruses, etc.), under conditions which promote binding,
separating the unbound sequences,
dissociating the nucleic acid-target element complexes,
amplifying the dissociated nucleic acids, so as to obtain a mixture enriched in ligands (aptamers), and
repeating the binding, separation, dissociation and amplification steps, for the desired number of times.

Among the abovementioned American patents:
U.S. Pat. No. 5,270,163 describes the initial principle of the SELEX method;
U.S. Pat. No. 5,580,737 describes an application of this method, comprising a counterselection step, to the selection of aptamers capable of discriminating between two substances of similar structure, namely caffeine and theophylline;
U.S. Pat. No. 5,660,985 describes an application of this method to the selection of modified aptamers containing modified nucleotides at the level of the pyrimidines (modification in the 5-position or in the 2′-position);
U.S. Pat. No. 5,789,157 describes an application of this method to the selection of aptamers capable of binding to proteins present at the surface of tissues and in particular of cells; this patent recommends in particular applying the SELEX method to specific targets, namely tissues and in particular tumor cells, which are considered to be targets more complex than those used in the other patents cited, and which are generally targets that have been molecularly identified (proteins, etc.). This U.S. Pat. No. 5,789,157 also specifies that it is possible to use a counter selection (or negative selection), before, during or after the implementation of the SELEX method. The negative selection more specifically described in U.S. Pat. No. 5,580,737 makes it possible to discriminate between tissue types which are different but nevertheless very similar. For example, negative selection can be used to identify ligands which exhibit a high specificity for tumor cells and not with respect to the corresponding normal cells. This patent also envisions the case where the nucleic acids which are ligands of a cell type which expresses a certain receptor can be counterselected with a cell line constructed in such a way that it does not express said receptor; however, the use of such a strategy can be difficult to implement if the protein induces a substantial phenotypic change in the cell;
U.S. Pat. No. 6,232,071 describes an application of this method to the selection of aptamers specific for tenascin C.

Surprisingly, the Applicant has found that it is possible to obtain aptamers specific for cell receptors, and more specifically for tumor markers, by carrying out the method known as SELEX on live target cells, under certain conditions.

Consequently, according to a first aspect, a subject of the invention is a method for identifying ligands or aptamers specific for a membrane receptor with tyrosine kinase activity (RPTK for receptor protein-tyrosine kinase) expressed in an activated form by cells (whatever the origin or the cause of the activation) or nonactivated form (preferably in an activated form), using a mixture of nucleic acids, which method comprises at least the following steps:
(a) bringing a mixture of nucleic acids into contact with cells not expressing said receptor protein-tyrosine kinase or expressing it in a nonactivated form (C_{N} cells), said cells having the same cell type as cells expressing the same receptor protein-tyrosine kinase but in an activated form, due to the existence of at least one mutation in the extracellular domain (C_{Te} cells);
(b) recovering a first subset S1 of nucleic acids which do not bind to the C_{N} cells, in step (a);
(c) bringing said first subset S1 into contact with Cᵢ cells, having the same cell type as the C_{Te} cells, but expressing said receptor protein-tyrosine kinase mutated in its intracellular part, said Cᵢ cells exhibiting a phenotype of the same type as that of the C_{Te} cells;
(d) recovering a second subset S2 of nucleic acids which do not bind to the Cᵢ cells in step (c);
(e) bringing the second subset S2 into contact with the C_{Te} cells;
(f) recovering the nucleic acids which bind to said C_{Te} cells, i.e. those exhibiting a high affinity with respect to the cells expressing said receptor protein-tyrosine kinase mutated in the extracellular domain (activated receptor), after dissociation of the cell-nucleic acid complexes;
(g) amplifying said nucleic acids with high affinity for the cells expressing said receptor protein-tyrosine kinase mutated in the extracellular domain (activated receptor), so as to obtain a mixture of nucleic acids, enriched in nucleic acids having a high affinity for said C_{Te} cells, and
(h) identifying the ligands or aptamers specific for the cells expressing receptor protein-tyrosine kinases (RPTKs) in an activated form, from the mixture obtained in (g).

Surprisingly, such a method, even though it comprises steps for excluding aptamers which bind to nonactivated forms of RPTK, makes it possible to select aptamers specific for RPTK, i.e., either aptamers capable of binding to said RPTK and of inhibiting the activity of said RPTK (activation of the kinase cascade), or aptamers capable only of binding to said RPTK (of advantage in imaging applications).

### DEFINITIONS

Receptor Protein-Tyrosine Kinases (RPTKs)

The receptor protein-tyrosine kinases (RPTKs) constitute a very large family of proteins. There are currently more than 90 known genes encoding protein tyrosine kinases (PTKs), in the human genome (Blume-Jensen P. et al., Nature, 2001, 411, 355-365): 58 encode transmembrane RPTKs divided up into 20 families and 32 encode cytoplasmic PKTs. Among the human receptor protein-tyrosine kinases (RPTKS) involved in cancers, mention may be made of the following families: EGFR (Epithelial Growth Factor Receptor), InsulinR (Insulin Receptor), PDGFR (Platelet-derived Growth Factor Receptor), VEGFR (Vascular Endothelial Growth Factor Receptor), FGFR (Fibroblast Growth Factor Receptor), NGFR (Nerve Growth Factor Receptor), HGFR (Hepatocyte Growth Factor Receptor), EPHR (Ephrin Receptor), AXL (Tyro 3 PTK), TIE (Tyrosine Kinase Receptor in endothelial cells), RET (Rearranged During Transfection), ROS (RPTK expressed in certain epithelial cells) and LTK (Leukocyte Tyrosine Kinase). In the subsequent text, the term “RPTK” used without other specification implies any receptor (activated or nonactivated; in an activated or nonactivated form).

Nonactivated RPTK

In its normal form, said receptor is not activated; activation is observed only after suitable stimulation of cells expressing said normal receptor (RPTK activated after stimulation).

RPTK Mutated in the Extracellular Domain (Activated Receptor)

In certain abnormal forms, due to mutations in the extracellular domain of the receptor protein-tyrosine kinase (one or more point mutations, insertions, deletions and/or rearrangements), a constitutive activation or an overexpression of the receptor is observed; such an activated receptor mutated in the extracellular portion is a constitutive activator of the kinase cascade.

RPTK Mutated in the Intracellular Domain

Such a receptor can activate certain intracellular cascades; it is not considered, for the purpose of the present invention, to be an activated receptor; on the other hand, it is included in the definition of receptors in or as an activated form.

RPTK in or as an Activated Form

The expression “receptor in or as an activated form” is intended to mean an RPTK which activates the kinase cascade, whatever the reason for this:
activation by stimulation with a growth factor (normal activation, under certain conditions),
constitutive activation or overexpression of the receptor, due to the existence of one or more mutations, either in the extracellular portion, or in the intracellular portion of said receptor.

Surprisingly, the specific conditions of the method according to the invention effectively make it possible to select and identify ligands or aptamers specific for the receptor protein-tyrosine kinase(s) preselected, i.e. which bind to said receptor, and in addition, among these, to select those capable of inhibiting said receptors in their activated form. In fact, the selection of the aptamers with C_{N}, C_{Te} and Cᵢ cells, as defined above, effectively makes it possible to obtain, in particular after repetition of steps a) to g), aptamers specific for cells expressing preselected receptor protein-tyrosine kinases (RPTKs).

In accordance with the invention, several cycles of steps (a) to (g) can advantageously be repeated using the mixtures enriched in ligands or aptamers from the preceding cycle, until at least one aptamer is obtained, the affinity of which, defined by its dissociation constant (Kd), can be measured and is suitable for pharmaceutical use.

Also in accordance with the invention:
the starting combinatorial library of nucleic acids advantageously consists of oligonucleotides comprising random sequences, of the same type as those described in the abovementioned SELEX patents. It contains at least 10² nucleic acids, preferably between 10⁹ and 10¹⁵ nucleic acids; the nucleic acids constituting said combinatorial library are preferably natural nucleic acid sequences (RNA or DNA) or modified nucleic acid sequences (for example, pyrimidines modified with a fluorine atom in the 2′-position of the ribose) consisting of random sequences comprising, respectively at their 5′ and 3′ ends, fixed sequences for PCR amplification, preferably the oligonucleotides of sequences SEQ ID NO: 1 and SEQ ID NO: 2, or a fragment of at least 8 nucleotides of these oligonucleotides. Said random sequences each contain between 10 and 1000 nucleotides, preferably 50 nucleotides.
the aptamers selected are defined by virtue of their primary sequence and by virtue of their secondary structure; the latter is either in hairpin loop form, or in the form of more complex structures (for example: pseudo-knot, triple helix, Guanine quartet, etc.). They advantageously consist preferably of nucleic acid sequences of 20 to 100 nucleotides.

Advantageously, the conditions described above make it possible to identify ligands or aptamers against molecular determinants (or markers) of pathologies, which can actually be effective under the very conditions of their future use, i.e. in vivo.

Thus, by selecting against a specific target, i.e. the C_{N}, Cᵢ and C_{Te} cells as defined above, aptamers are effectively obtained which specifically recognize cells expressing a preselected receptor protein-tyrosine kinase in particular in its activated form.

According to an advantageous embodiment of said method, the identification of the ligands or aptamers specific for the C_{Te} cells according to step (h) comprises an evaluation of the biological activity of said aptamers on said C_{Te} cells.

The biological activities, which are advantageously evaluated, depend on the receptor selected; they are in particular the following:
(a) inhibition or activation of the autophosphorylation of the receptor (RPTK),
(b) inhibition or activation of the kinase activation cascade,
(c) inhibition of the phosphorylation of the normal RPTK of normal cells (C_{N} cells) activated by suitable stimulation (appropriate growth factor, for example),
(d) reversion of the phenotype associated with activation of the RPTK.

For the purpose of the present invention, the following terms are considered to be equivalent: nucleic acid fragment, oligonucleotide, ligand or aptamer.

According to a second aspect, a subject of the present invention is ligands or aptamers, characterized in that they are specific for cells expressing a receptor protein-tyrosine kinase (RPTK) in an activated or nonactivated form (preferably in an activated form), in particular an RPTK mutated in the extracellular domain, and can be identified by means of the method for identifying aptamers, as defined above.

According to an advantageous embodiment of said aptamer, it is specific for cells expressing a receptor protein-tyrosine kinase (RPTK) in an activated or nonactivated form, selected in particular from the group consisting of the following membrane receptors, given by way of nonlimiting examples: EGFR (Epithelial Growth Factor Receptor), InsulinR (Insulin Receptor), PDGFR (Platelet-derived Growth Factor Receptor), VEGFR (Vascular Endothelial Growth Factor Receptor), FGFR (Fibroblast Growth Factor Receptor), NGFR (Nerve Growth Factor Receptor), HGFR (Hepatocyte Growth Factor Receptor), EPHR (Ephrin Receptor), AXL (Tyro 3 PTK), TIE (Tyrosine Kinase Receptor in endothelial cells), RET (Rearranged During Transfection), ROS(RPTK expressed in certain epithelial cells) and LTK (Leukocyte Tyrosine Kinase).

According to an advantageous arrangement of this embodiment, said aptamer recognises in particular the Ret receptor activated by mutation at a cysteine located in the extracellular domain, preferably at codons 609, 611, 618, 620 or 634.

According to a preferred mode of this arrangement, said aptamer can be identified by means of a method, as defined above, which comprises:
(a) bringing a mixture of nucleic acids into contact with C_{N} cells not expressing any Ret receptor in an activated form,
(b) recovering a first subset S1 of nucleic acids which do not bind to said C_{N} cells, in step (a),
(c) bringing said first subset S1 into contact with Cᵢ cells expressing a Ret receptor, mutated in its intracellular domain, in particular the mutated receptor Ret^{M918T},
(d) recovering a second subset S2 of nucleic acids which do not bind to said Cᵢ cells,
(e) bringing the second subset S2 into contact with C_{Te} cells expressing a Ret receptor activated by mutation in the extracellular domain, which receptor is selected from the group consisting of mutated Ret receptors carrying a mutation on one of the cysteines located in the extracellular domain, preferably at Cys609, Cys611, Cys618, Cys620 or Cys634, preferably the Ret^{C634Y} receptor,
(f) recovering the nucleic acids bound to said C_{Te} cells, i.e. exhibiting both a high affinity and a binding specificity for the cells expressing a mutated Ret receptor (activated receptor) as defined in step (e),
(g) amplifying said nucleic acids obtained in step (f), so as to obtain a mixture of nucleic acids, enriched in nucleic acids having a high affinity for the C_{Te} cells,
(h) repeating steps (a)-(g), until at least one aptamer is obtained, the affinity of which for the C_{Te} cells, defined by its dissociation constant (Kd), is measurable and suitable for a pharmacological activity, and
(i) identifying the aptamers specific for the cells expressing a Ret receptor in its activated form, selected from the mixture obtained in (h).

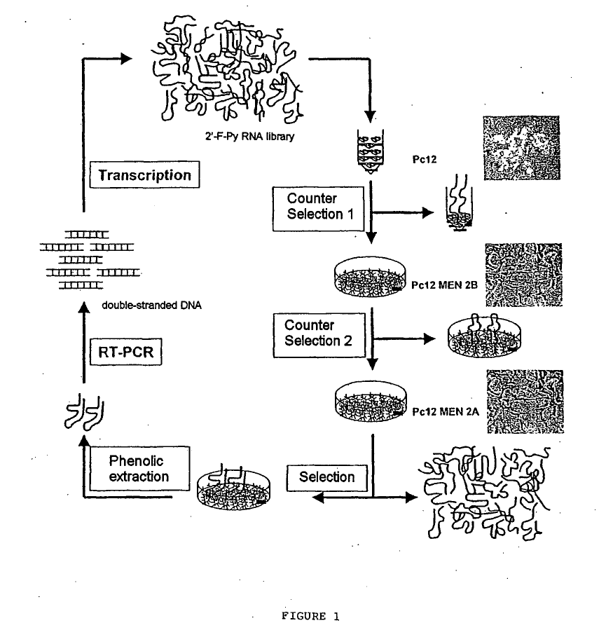

The cycle for obtaining the aptamers, according to the invention, applied to the Ret receptor, is illustrated in FIG. 1.

The Ret (rearranged during transfection) oncogene encodes an abnormal form of a receptor-type surface protein of the tyrosine kinase family; this protooncogene is located on chromosome 10q11.2. Mutations in the Ret protooncogene are associated with disparate diseases, in particular Hirschsprung's disease and multiple endocrine neoplasia type II (or MEN 2), which includes MEN type 2A (MEN 2A), MEN type 2B (MEN 2B) and familial medullary thyroid cancer (or FMTC). MEN 2A is characterized by a medullary thyroid carcinoma, a pheochromocytoma and parathyroid hyperplasia (primary hyperparathyroidism). MEN 2B is characterized by a particularly aggressive form of medullary thyroid cancer, a pheochromocytoma, multiple mucosal neurogliomas and intestinal ganglioneuromatosis. A truncated form of ret encodes an intracellular protein associated with papillary thyroid cancer (PTC).

Oncogenes are mutated forms of protooncogenes, which are normal proteins, the function of which is to control cell growth and division, in particular after activation by appropriate growth factors (such as, for example, GDNF for the protooncogene encoding the Ret receptor). Certain mutations of these protooncogenes result in forms of these proteins which are permanently active, even in the absence of stimulation by the usual growth factor(s) (deregulation). This constitutive (permanent) activation results in a permanent stimulation of cell growth and division, and, in the end, in cancerization. The mutated form of the protooncogene is then referred to as a tumor-activating oncogene. Oncogenes can induce a cancerization, for example, by overproduction of growth factors, or by inundation of the cell with replication signals, or by uncontrolled stimulation of intermediate pathways, or by disorganized cell growth linked to a high level of transcription factors. Certain oncogenes are transmitted from generation to generation, when the protooncogene mutates in the germinal cells. This implies an inherited and dominant tumor predisposition. For example, multiple endocrine neoplasia type II (or MEN 2) is the result of a germinal transmission of the activated Ret oncogene.

Mutations in exons 10 and 11 of the protooncogene are observed in more than 95% of cases of MEN 2A and in more than 80% of cases of MTC; most of these mutations are located at five conserved cysteines located in the extracellular domain (codons 609, 611, 618, 620 and 634). These mutants of the Ret receptor spontaneously form active homodimers at the surface of the cell, which induce morphological and biochemical changes, resulting in a pheochromocytoma-type phenotype, dependent on the Ret receptor in MEN2 syndromes; mutations at codon 634 are the most frequent in MEN 2A. The activation of the Ret receptor can be followed by means of the phosphorylation cascade (Jhiang S M, Oncogene, 2000, 19, 5590-5597; Califano D et al., PNAS, 1996, 93, 7933-7937).

As regards MEN 2B, a point mutation in exon 16 of the protooncogene at codon 918 of the ret gene has been identified in approximately 95% of cases; this mutation leads to the substitution of a threonine with a methionine, in the catalytic domain of the Ret receptor. This mutation results in activation of the receptor in the form of a monomer.

Surprisingly, the selection of the aptamers with C_{N}, C_{Te} and Cᵢ cells, as defined above, effectively makes it possible to obtain, after repetition of steps a) to g), aptamers specific for the human form of Ret receptors, and in particular Ret receptors in their activated form, for example the mutated Ret^{C634Y} receptors, expressed by the C_{Te} cells, normal receptors, which may or may not be activated, or receptors mutated in the intracellular portion (see definitions).

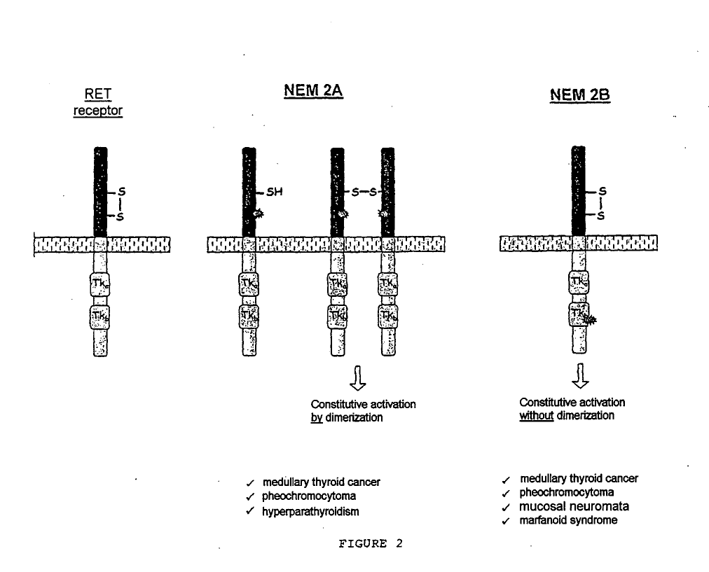

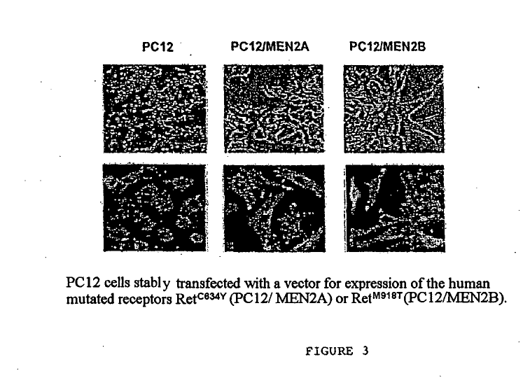

In accordance with the invention:
the C_{N} cells are in particular wild-type PC12 cells (reference ECACC No. 88022) or wild-type NIH 3T3 cells (reference ECACC No. 93061524),
the Cᵢ and C_{Te} cells are obtained by introducing an oncogene bearing a mutation, respectively intracellular or extracellular (FIG. 2), in C_{N} cells in culture in such a way that the latter express the oncogene; in the context of the invention, a similar transformed phenotype (FIG. 3) is obtained in the two cases. The PC12 cells change from a relatively non-adherent round shape to an elongated shape with pseudoneurites which exhibit good adhesion capacities (Califano D et al., PNAS, 1996, 93, 7933-7937).

It is then possible to carry out the selection and counterselection on virtually identical phenotypes which differ only by virtue of the cellular location of the oncogenic mutation (FIG. 1); more specifically, the Cᵢ cells obtained are called PC12/MEN 2B (or NIH/MEN 2B) cells and the C_{Te} cells obtained are called PC12/MEN 2A (or NIH/MEN 2A) cells.

Advantageously, the conditions described above make it possible to identify ligands or aptamers against molecular determinants (or markers) of pathologies, which will actually be effective under the very conditions of their future use, i.e. in vivo.

Thus, by selecting a specific target, i.e. the C_{N}, Cᵢ and C_{Te} cells, as defined above, aptamers are effectively obtained which specifically recognize the cells expressing the human form of the Ret receptor in an activated or nonactivated form, preferably in an activated form.

The identification of an aptamer specific for cells expressing a human form of the Ret receptor in its activated or nonactivated form, as defined above, advantageously comprises an additional step (j) consisting in evaluating its biological activity on said C_{Te} cells.

The biological activities which are advantageously evaluated are as follows:
(a) inhibition or activation of the autophosphorylation of the Ret receptor,
(b) inhibition or activation of the Erk kinase activation cascade, Erk being an intracellular protein downstream of Ret in the cascade,
(c) inhibition of the phosphorylation of the normal Ret receptor of PC12 cells activated with GDNF and reversion of the phenotype which is associated therewith,
(d) reversion of the phenotype associated with the activation of the RPTK overexpressing the oncogene (Ret^{C634Y}).

Said aptamer can be obtained by means of a method of identification as specified above, and is selected from the group consisting of the aptamers of formula (I):

R₁-R-R₂ (I)

in which:
R₁ represents 5′ GGGAGACAAGAAUAAACGCUCAA 3′ (SEQ ID NO:1) or a fragment of 1 to 23 nucleotides of said SEQ ID NO:1;
R₂ represents 5′ AACGACAGGAGGCUCACAACAGGA 3′ (SEQ ID NO:2) or a fragment of 1 to 24 nucleotides of said SEQ ID NO:2, and
R represents a random sequence of 10 to 1000 nucleotides, preferably of 50 nucleotides.

According to an advantageous arrangement of this embodiment, R is preferably selected from the following sequences:

| | | |
|---|---|---|
| D4 | 5′GCGCGGGAAUAGUAUGGAAGGAUACGUAUACCGUGCAAUCCAGGGCAACG 3′ | (SEQ ID NO:3) |
| D12 | 5′GGGCUUCAUAAGCUACACCGGCCAACGCAGAAAUGCCUUAAGCCCGAGUU 3′ | (SEQ ID NO:4) |
| D14 | 5′GGCCAUAGCGCACCACCAAGAGCAAAUCCCUAAGCGCGACUCGAGUGAGC 3′ | (SEQ ID NO:5) |
| D20 | 5′GGGCCAAUCGAAGCCGGUAAUUCCCAAACUAACGUGCAAACUGCACCCGC 3′ | (SEQ ID NO:6) |
| D24 | 5′GCGGUAUGUAGGGAAUAGCACUUUUUUUGCGUAUACCUACACCGCAGCG 3′ | (SEQ ID NO:7) |
| D30 | 5′AGGCGAGCCCGACCACGUCAGUAUGCUAGACAACAACGCCCGCGUGGUAC 3′ | (SEQ ID NO:8) |
| D32 | 5′CCCCGCUUUUUGACGUGAUCGAACGCGUAUCAGUAACGUCAGCAGUCGAGC 3′ | (SEQ ID NO:9) |
| D33 | 5′CAAAGCGUGUAUUCUCGUGAGCCGACCAUCGUUGCGAACAUCCCCGGAACG 3′ | (SEQ ID NO:10) |
| D42 | 5′GACCCGUAUGAAGGUGGCGCAGGACACGACCGUCUGCAAUGAGCGAGC 3′ | (SEQ ID NO:11) |
| D60 | 5′CCGACCUGUACAGCAGUUAGUUACACGUUUGAAACAACCGGCGUUCGAGC 3′ | (SEQ ID NO:12) |
| D76 | 5′GGCUUACACGGAGAAACAAGAGCGGCCCAAACUUGAUUGACAGUGGCC 3′ | (SEQ ID NO:13) |
| D71 | 5′GGCCCUUAACGCAAAAACGAAGGAUCAUCGAUUGAUCGCCUUAUGGGCU 3′ | (SEQ ID NO:14) |
| D87 | 5′CCGCGGUCUGUGGGACCCUUCAGGAUGAAGCGGCAACCCAUGCGGGCC 3′ | (SEQ ID NO:15) |

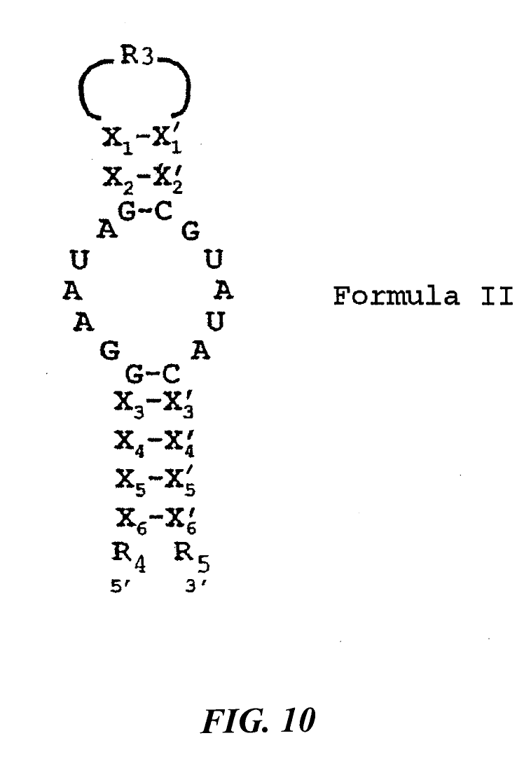

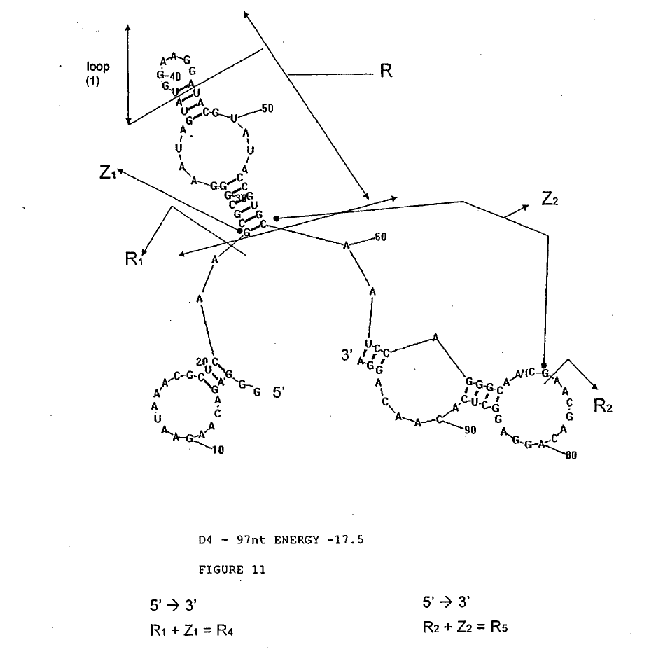

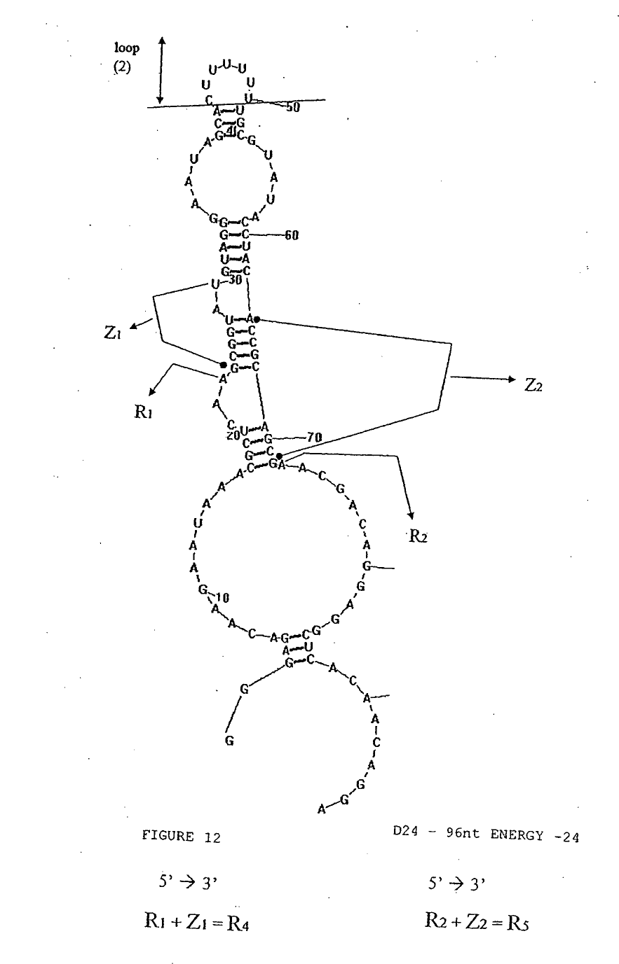

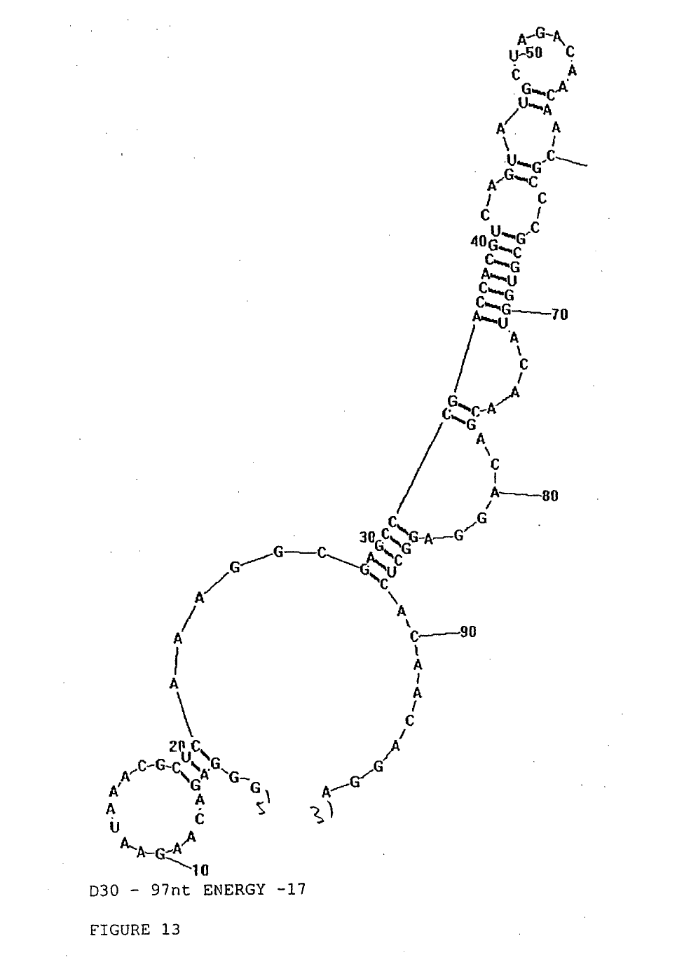

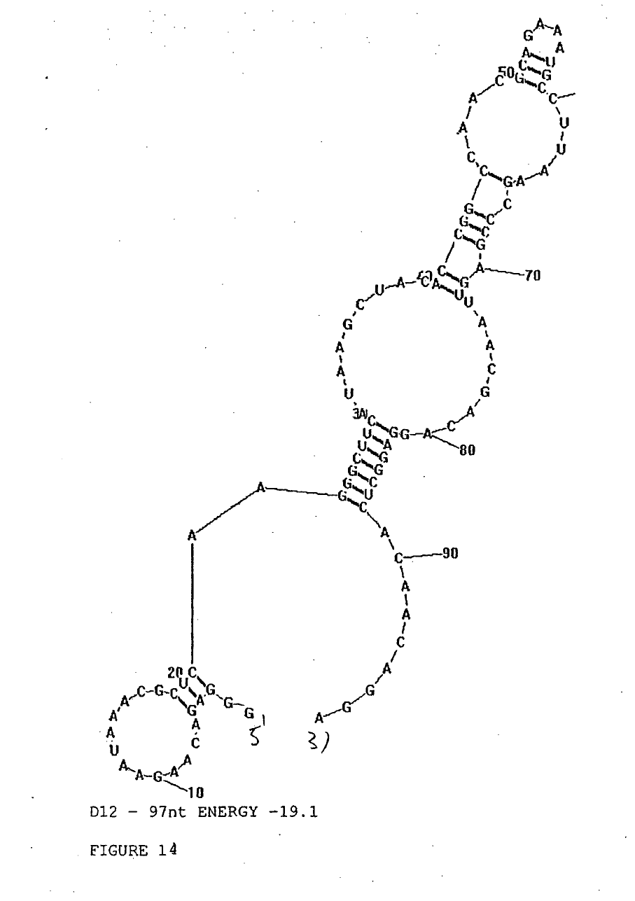

The preferred aptamers in which the Rs are as defined above are represented by the sequences SEQ ID NO:22 (D4; FIG. 11), SEQ ID NO:25 (D24; FIG. 12), SEQ ID NO:31 (D30; FIG. 13), SEQ ID NO:32 (D12; FIG. 14), SEQ ID NO:33 (D71; FIG. 15).

In accordance with the invention, in said aptamers, the riboses of the purines bear, as is the case in natural RNA, a hydroxyl (OH) function on the carbon in the 2′-position, while the riboses of the pyrimidines bear a fluorine atom on the carbon in the 2′-position. This modification of the 2′-position is known to confer on the nucleic acids a greater resistance with respect to nucleases.

The sequences of the primers used to carry out step (g) consisting in amplifying the mixture of nucleic acids of formulae R₁-R-R₂, in which R₁ represents SEQ ID NO:1 and R₂ represents SEQ ID NO:2, are advantageously as follows:

Sense primer (primer P10):

(SEQ ID NO:16)

TAATACGACTCACTATAGGGAGACAAGAATAAACGCTCAA;
Antisense primer (primer P30):

(SEQ ID NO:17)

TCCTGTTGTGAGCCTCCTGTCGTT.

The prediction of secondary and tertiary structure of the aptamers selected is carried out using the RNAstructure software written by David H. Mathews: http://rna.chem.rochester.edu. The algorithm used by this software is based on the searches described in the publication: D. H. Mathews et al., J. Mol. Biol., 1999, 288, 911-940. The same predictions can be obtained using the mfold algorithm, available of the site of the Michael Zuker laboratory: http://bioinfo.math.rpi.edu/˜zukerm/. The algorithm used by this software is also based on the searches described in the publication D. H. Mathews et al., mentioned above.

Among the aptamers described above, some have a common structure, defined by formula II below:

5′R₄X₆X₅X₄X₃GGAAUAGX₂X₁R₃X′₁X′₂CGUAUACX′₃X′₄X′₅X′₆R₅ 3′ (II),

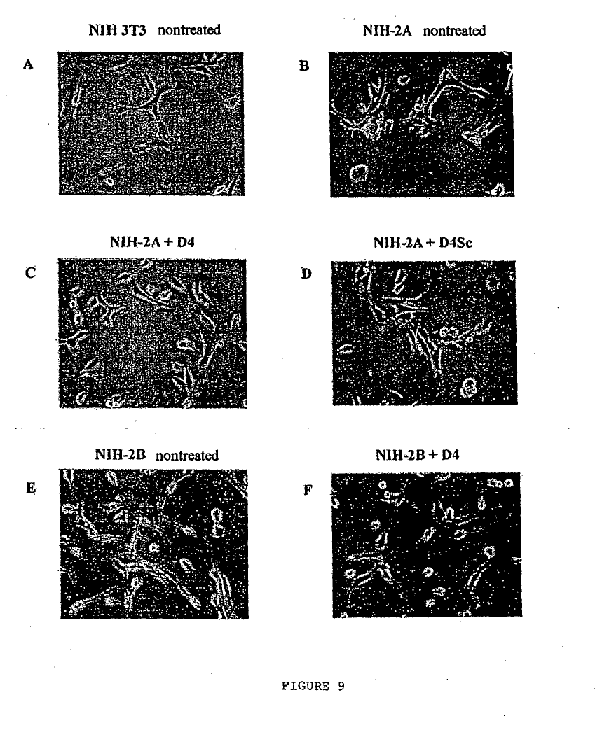

the secondary structure of which is represented in FIG. 10, and in which:
the riboses of the purines bear an OH group in the 2′-position and the riboses of the pyrimidines bear a fluorine atom in the 2′-position,
R₃ is present or absent and represents an apical bulge (or loop) comprising:
a linear or branched carbon chain selected from the group consisting of C₆-C₃₀ alkyl groups or C₆-C₃₀ aryl groups;
a polymer such as PEG or PEI, or the like;
functional groups such as biotin, streptavidin, peroxidase, etc.;
other molecules of interest such as, for example, active ingredients, labeling tags, in particular fluorescent tags, or chelating agents for radioisotopes;
a natural (DNA or RNA) or modified nucleotide sequence (for example: 2′-fluoro, 2′-O-methyl, PNA, LNA, etc.); preferably, R₃ represents the following bulges or loops (1) to (4):

| | |
|---|---|
| loop (1): 5′ UGGAAGGA 3′ | (SEQ ID NO:29) |
| loop (2): 5′ CUUUUUU 3′ | (SEQ ID NO:30) |
| loop (3): 5′ GNPuA 3′ | |
| loop (4): 5′ UNCG 3′, | |

in which the riboses of the purines bear a hydroxyl function on the carbon in the 2′-position, while the riboses of the pyrimidines bear a fluorine atom on the carbon in the 2′-position,
X₁, X′₁, X₂, X′₂, X₃, X′₃, X₄, X′₄, X₅, X′₅, X₆ and X′₆ represent Py or Pu with, preferably:
   X₁-X′₁ corresponding to C-G, A-U, G-C or U-A
   X₂-X′₂ corresponding to C-G, A-U, G-C or U-A
   X₃-X′₃ corresponding to C-G, A-U, G-C or U-A
   X₄-X′₄ corresponding to C-G, A-U, G-C or U-A
   X₅-X′₅ corresponding to C-G, A-U, G-C or U-A
   X₆-X′₆ corresponding to C-G, A-U, G-C or U-A
   N corresponding to G or C or A or U,
   Pu corresponding to G or A, in which the riboses bear an OH group in the 2′-position (natural RNA chemistry), Py corresponds to U or C, in which the riboses bear a fluorine atom in the 2′-position, and
R₄ and R₅ are present or absent and represent:
   a natural (DNA or RNA) or modified nucleotide sequence (for example: 2′-fluoro, 2′-O-methyl, PNA, LNA, etc.), comprising between 1 and several thousand nucleotides, preferably between 1 and 39 nucleotides; a part of said nucleotide sequence or said sequence preferably comprising one of the following sequences:

R₄:
5′-R₁-Z₁-3′, with Z₁ = G:

(SEQ ID NO:18)

5′ GGGAGACAAGAAUAAACGCUCAAG 3′
or
5′-R₁-Z₁-3′, with Z₁ = GCGGUAU (SEQ ID NO:26):

(SEQ ID NO:19)

5′ GGGAGACAAGAAUAAACGCUCAAGCGGUAU,
and
R₅:
5′-Z₂-R₂-3′, with Z₂ = CAAUCCAGGGCAACG (SEQ ID
NO:27):

(SEQ ID NO:20)

5′ CAAUCCAGGGCAACGAACGACAGGAGGCUCACAACAGGA 3′
or
5′-Z₂-R₂-3′, with Z₂ = ACCGCAGCG (SEQ ID NO:28):

(SEQ ID NO:21)

5′ ACCGCAGCGAACGACAGGAGGCUCACAACAGGA 3′,

a linear or branched carbon chain selected from the group consisting of C₆-C₃₀ alkyl groups or C₆-C₃₀ aryl groups;
a polymer such as PEG or PEI, or the like,
functional groups such as biotin, streptavidin, peroxidase, etc.,
other molecules of interest such as, for example, active ingredients, labeling tags, in particular fluorescent tags, or chelating agents for radioisotopes.

Among the aptamers having a structure as defined in formula II and in FIG. 10, differences in properties as specified hereinafter are observed:

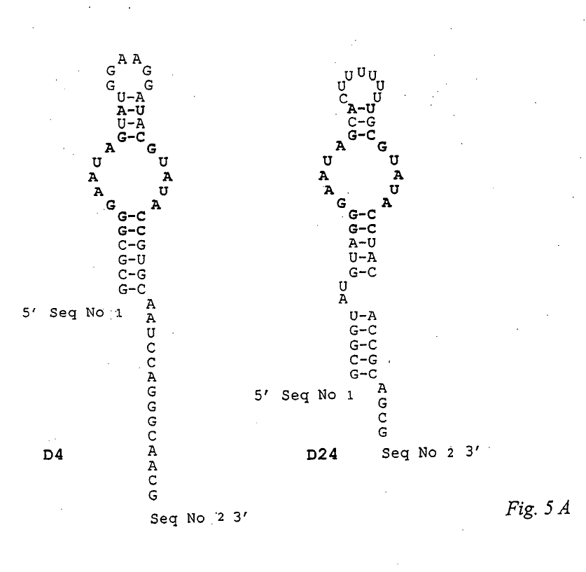

The structure of formula II, in which R₃, R₄ and R₅ are absent, is sufficient for binding to the Ret^{C634Y} receptor, whereas the structure of formula II, in which R₃, R₄ and R₅ are present, exhibit either binding properties only, or both binding and inhibiting properties; these properties varying as a function of R₃, R₄ and R₅:
When R₃ represents 5′ UGGAAGGA 3′ (SEQ ID NO:29: loop (1)), R₄ represents SEQ ID NO:18 and R₅ represents SEQ ID NO:20, the aptamer exhibiting such a structure (family D4) has both properties of binding to said Ret receptor in its activated form and properties of inhibition of the activity of said receptor. The secondary structure of this product is represented in FIG. 11; it involves the products of family D4, in which R₄ and R₅ comprise a sufficient number of nucleotides resulting in inhibition of the activity of the Ret receptor. A preferred aptamer corresponding to this definition comprises, from 5′ to 3′, successively the following sequences: SEQ ID NO:1+SEQ ID NO:3+SEQ ID NO:2 (SEQ ID NO:22) (D4: FIG. 5A and FIG. 11), with reference to formula I.
When R₃ represents 5′ CUUUUUU 3′ (SEQ ID NO:30: loop (2)), 5′GNPuA 3′ (loop 3) or 5′UNCG 3′ (loop 4), R₄ comprises from 1 to 30 nucleotides selected from SEQ ID NO:19 or from 1 to 24 nucleotides selected from SEQ ID NO:18 and R₅ comprises from 1 to 33 nucleotides selected from SEQ ID NO:21 or from 1 to 39 nucleotides selected from SEQ ID NO:20, the aptamer exhibiting such a structure has only properties of binding to said Ret receptor in its activated form, and in particular to the Ret receptor mutated in its extracellular domain. When R₃ represents 5′ CUUUUUU 3′ (loop 2), R₄ represents SEQ ID NO:19 and R₅ represents SEQ ID NO:21, the secondary structure of this product is represented in FIG. 12; the preferred product is represented by SEQ ID NO:25 and belongs to the family D24, in which R₄ and R₅ comprise at least one nucleotide. This aptamer comprises successively from 5′ to 3′: SEQ ID NO:1+SEQ ID NO:7+SEQ ID NO:2, with reference to formula I.
When R₃ represents 5′ UGGAAGGA 3′ (loop 1), and in the absence of R₄ and of R₅, the aptamer exhibiting such a structure has only properties of binding to said Ret receptor in its activated form. A preferred aptamer corresponding to the latter definition corresponds to a part of SEQ ID NO:3 of family D4 (SEQ ID NO:23).

The differences between the definitions of R₁ and R₂ in formula I R₁-R-R₂ and of R₄ and R₅ in formula II come from the non-superposition between the consensus sequence of FIG. 10 and the definition of R (random sequences) in formula I.

FIGS. 5A and 10 to 12 show the links between the two formulae (I and II).

The ligands or aptamers according to the invention can advantageously be used in the following applications:
as diagnostic reagent: both in vitro (diagnostic kits; histological marker; chips) and in vivo (contrast agent for imaging; radiopharmaceuticals). In fact, the aptamers according to the invention which have an ability to bind specifically to a Ret receptor in its activated or nonactivated form are suitable for the detection of cells expressing said receptor in activated or nonactivated form; in particular, when the Ret receptor is mutated in its extracellular domain, said aptamers are particularly suitable for the detection of abnormal cells expressing said receptor; when the receptor in its activated form is not mutated, a difference in level of expression of the protein will then be followed;
as medicament, in particular as anticancer agent; in particular as regards the aptamers which exhibit both an ability to bind to the Ret receptor and an inhibitory action with respect, in particular, to the mutated Ret^{C634Y} receptor.

In fact, surprisingly:
some of the aptamers selected have only a binding activity and will preferably be used as diagnostic reagents;
other aptamers selected also exhibit an inhibitory activity on the oncogenic transformation mediated by the human Ret oncogene mutated in particular at a cysteine in the extracellular domain, and an inhibitory effect on the activity of the normal Ret protein after stimulation by its own ligand (family D4 or sequences derived from formula II with chemical modifications). The aptamers exhibiting such an activity may be used either as diagnostic agents or as medicaments. In order to characterize them, four tests can advantageously be carried out, as specified below:
(a) inhibition or activation of the autophosphorylation of Ret,
(b) inhibition or activation of the Erk kinase activation cascade, Erk being an intracellular protein downstream of Ret in the cascade,
(c) inhibition of Ret phosphorylation activated by GDNF,
(d) reversion of the phenotype associated with the activation of Ret (in particular, Ret^{C634Y})

A subject of the present invention is also a reagent for diagnosing a tumor, characterized in that it consists of at least one aptamer as defined above.

According to an advantageous embodiment of said reagent, it corresponds to an aptamer of formula II, as defined above:

5′R₄X₆X₅X₄X₃GGAAUAGX₂X₁R₃X′₁X′₂CGUAUACX′₃X′₄X′₅X′₆R₅3′,

in which R₃, R₄ and R₅ are absent.

According to an advantageous arrangement of this embodiment, said reagent corresponds to an aptamer of sequence:

| | |
|---|---|
| 5′GUAGGGAAUAGCACGUAUACCUAC3′, | (SEQ ID NO:24) |

in which X₁-X′₁=A-U, X₂-X′₂=C-G X₃-X′₃=G-C, X₄-X′₄=A-U, X₅-X′₅=U-A and X₆-X′₆=G-C.

According to another advantageous embodiment of said reagent, it corresponds to an aptamer of formula II, in which R₃ represents 5′ CUUUUUU 3′ (loop (2)), R₄ represents the sequence SEQ ID NO:19 and R₅ represents the sequence SEQ ID NO:21; this aptamer corresponds to SEQ ID NO:25, and comprises successively from 5′ to 3′, with reference to formula I: SEQ ID NO:1+SEQ ID NO:7+SEQ ID NO:2, as specified above.

A subject of the present invention is also a reagent for diagnosing or detecting the Ret receptor in an activated or nonactivated form, characterized in that it consists of at least one aptamer as defined above.

A subject of the present invention is also a medicament, characterized in that it comprises an aptamer as defined above which has both an ability to bind to an RPTK receptor and an inhibitory action with respect to said receptor in an activated form.

A subject of the present invention is also a medicament for use in the treatment of a tumor, characterized in that it comprises an aptamer as defined above, which has both an ability to bind to an activated RPTK receptor, and in particular to a receptor mutated in the extracellular domain, and in particular to the Ret receptor mutated, for example, at one of the cysteines located in the extracellular domain (codons 609, 611, 618, 620 and 634), and an inhibitory action with respect to this activated receptor.

According to an advantageous embodiment of said medicament, it corresponds to an aptamer of the aptamer family D4, as defined above.

A subject of the present invention is also a pharmaceutical composition, characterized in that it comprises an aptamer as defined above, which has both an ability to bind to an RPTK receptor and an inhibitory action with respect to said receptor in its activated form.

A subject of the present invention is also a pharmaceutical composition, characterized in that it comprises:
an aptamer as defined above, which has both an ability to bind to an RPTK receptor mutated in the extracellular domain, and in particular to the Ret receptor mutated, for example, at one of the cysteines located in the extracellular domain (codons 609, 611, 618, 620 and 634), and an inhibitory action with respect to this mutated receptor,
another anticancer molecule, and
at least one pharmaceutically acceptable vehicle.

A subject of the present invention is also the use of an aptamer which has both an ability to bind to an RPTK receptor and possibly an inhibitory action with respect to this RPTK receptor, for screening products which interact with the RPTK receptor and which may or may not inhibit it.

A subject of the present invention is also the use of an aptamer which has both an ability to bind to an RPTK receptor in its activated form, and in particular to the Ret receptor mutated at one of the cysteines located in the extracellular domain (codons 609, 611, 618, 620 and 634), and possibly an inhibitory action with respect to this mutated RPTK receptor, for screening products which interact with the RPTK receptor and which may or may not inhibit it.

A subject of the present invention is also a method for screening products which interact with an RPTK receptor or targets which form a complex with the RPTK (in an activated or nonactivated form), which method is characterized in that it comprises:
bringing cells expressing RPTKs in an activated or nonactivated form into contact with the substance to be tested,
adding, under suitable conditions, an optionally labeled aptamer, as defined above, before the substance to be tested, at the same time or after it, and
evaluating the competitive binding between the aptamer and the substance to be tested (for example: by measuring radioactivity, fluorescence, luminescence, surface plasmon resonance, BRET, FRET, or any other technique for demonstrating a molecular interaction).

In accordance with the invention, after identification of the substances which bind competitively with the aptamer to the cells exhibiting RPTKs in an activated form, the effect of these substances on the biological activity of said cells can be evaluated in order to find substances which inhibit or activate said biological activities of the cells expressing RPTKs in an activated form.

Besides the above arrangements, the invention also comprises other arrangements, which will emerge from the following description, which refers to examples of implementation of the method which is the subject of the present invention and also to the attached drawings, in which:

FIG. 1: diagrammatic selection of aptamers specific for PC12 MEN 2A cells:
steps a) and b): counterselection

A combinatorial library of 2′-F-Py RNAs is incubated with wild-type PC12 cells (PC12 wt) in suspension; the sequences not bound are recovered by centrifugation and incubated with PC12 MEN 2B cells; the sequences not bound, present in the supernatant, are recovered and incubated with PC12 MEN 2A cells.
step c): selection

The unbound sequences are removed by means of several washes of the cells and the bound sequences are recovered by extraction with phenol.
steps d) and e): amplification

The sequences selected are amplified by RT-PCR and in vitro transcription before a further selection cycle.

FIG. 2: structure of the various Ret receptors: normal Ret; MEN 2A Ret (Ret^{C634Y}); MEN 2B Ret (Ret^{M918T}).

FIG. 3: representation of the phenotype of PC12 cells stably transfected with an expression vector containing the sequence encoding the human mutated receptor Ret^{C634Y} (PC12 MEN 2A) or the mutated receptor Ret^{M918T} (PC12/EN 2B).

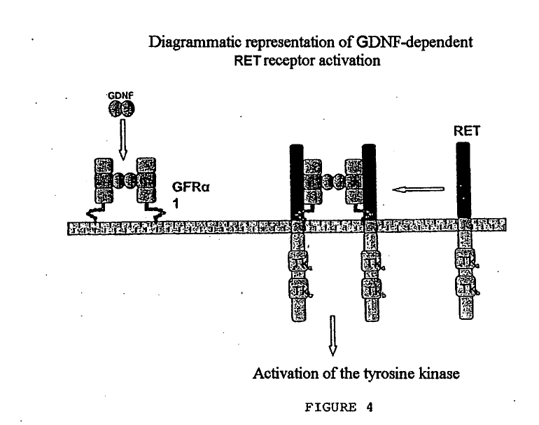

FIG. 4: diagrammatic representation of GDNF-dependent Ret receptor activation.

FIG. 5 (A): comparison of the prediction of the secondary structure of the D4 and D24 aptamers. The secondary structure prediction is carried out using the RNAstructure software written by David H. Mathews, http://rna.chem.rochester.edu. The algorithm is based on the searches described in D. H. Mathews et al. (Journal of Molecular Biology, 1999, 288, 911-940, mentioned above).

The same predictions can be obtained using the mfold algorithm, available on the site http://bioinfo.math.rpi.edu/˜zukerm/. The latter algorithm is based on the searches described in the publication in the name of D. H. Mathews et al., mentioned above. The consensus structure is in bold characters. (B): curve of binding of the D4 aptamer with PC12 MEN 2A cells; the D4 aptamer is radiolabeled with ³²P and incubated at various concentrations with cell monolayers. After several washes, the bound aptamer is quantified. The background noise is taken into account by subtracting, for each point obtained, the value obtained with a destructured D4 aptamer (D4Sc) having a scrambled sequence (i.e. containing the same nucleotides, but in a different order). A Scatchard analysis (insert) is used to evaluate the binding constant and the number of targets.

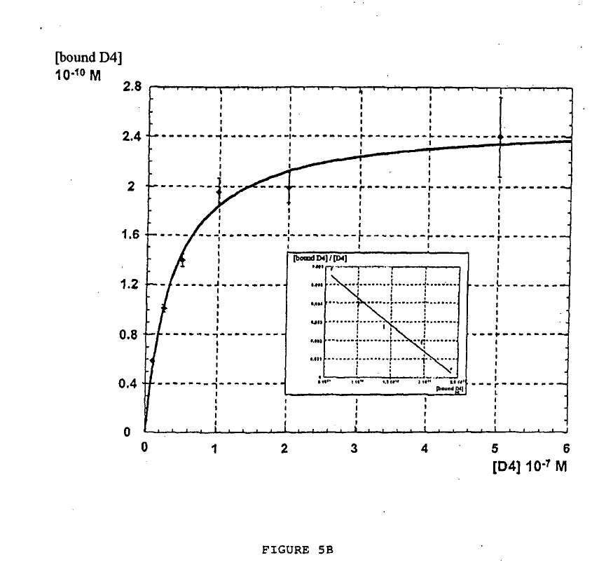

FIG. 6: effects of the various selected aptamers on the activity of the Ret^{C634Y} receptor: (A) the PC12 MEN 2A cells are either nontreated, or treated for 16 hours with 150 nM of the aptamer indicated or of the starting RNA pool (combinatorial library); (B) the PC12 MEN 2A cells are treated for one hour with increasing doses of D4 (product of formula II) (left) or with 200 nM of product D4 for the incubation times indicated (right). The cell lysates are subjected to analysis by immunoblotting with anti-Ret (Tyr-phosphorylated) antibodies or anti-(phospho) Erk antibodies, as indicated. In order to confirm equal load, the blotting membranes are subjected to a further analysis in the presence of the abovementioned anti-total Ret and anti-total Erk antibodies. The nontreated control cells are indicated by a “C”. The phosphorylation values, taking the value 1 for the control, were calculated using the NIH Image program, based on the sum of the two bands specific for Erk. The standard deviations are obtained from four independent experiments.

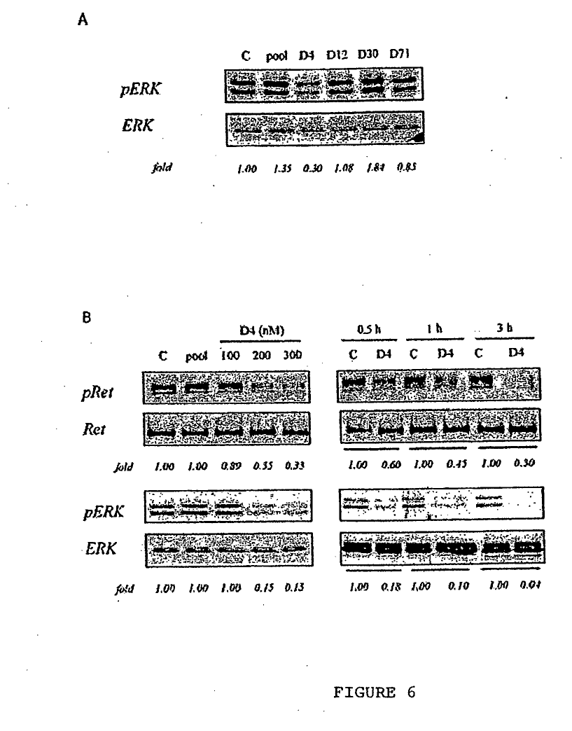

FIG. 7: effect of the D4 aptamer on the activity of the wild-type Ret receptor (Ret^{wt}) and of the mutated Ret^{M918T} receptor. (A) The PC12 cells transfected so as to stably express the nonmutated Ret receptor (PC12/wt) are treated for 10 min with GDNF (50 ng/ml) and soluble GFRα1 (1.6 nM), or 5 min with NGF (100 ng/ml) and also with, simultaneously, 200 nM either of D4 aptamer or of the starting RNA pool. (B) The PC12 MEN 2B cells are serum-deprived for 6 hours and then treated for 1 hour with 200 nM of D4 aptamer or of starting RNA pool. The cell lysates are analyzed by immunoblotting with the following antibodies: anti-Ret (Tyr-phosphorylated) antibodies or anti-(phospho) Erk antibodies, as indicated. The standard deviations are obtained from five independent experiments.

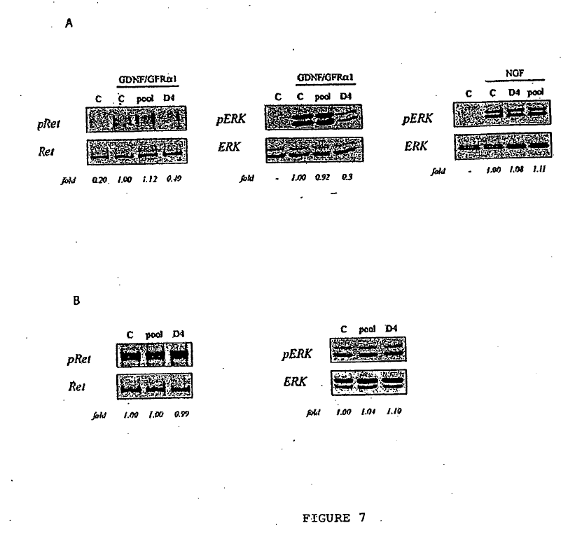

FIG. 8: the D4 aptamer inhibits the differentiation of PC12 cells transfected so as to stably express the nonmutated Ret receptor and the GFRα1 coreceptor (PC12-α1/wt) induced by GDNF. The cells are either nonstimulated (A) or stimulated with GDNF alone (B) or stimulated with GDNF in the presence of the D4 aptamer or of the destructured D4 aptamer (D4Sc) (C and D, respectively). After treatment for 48 hours with GDNF, the percentage extension of the processes is calculated. The data are expressed as the percentage of cells comprising processes relative to the total number of cells counted. Each experiment is repeated at least three times (E) and the cell lysates are analyzed by immunoblotting with anti-VGF antibodies, VGF being a marker for differentiation induced by GDNF-induced Ret activation (F).

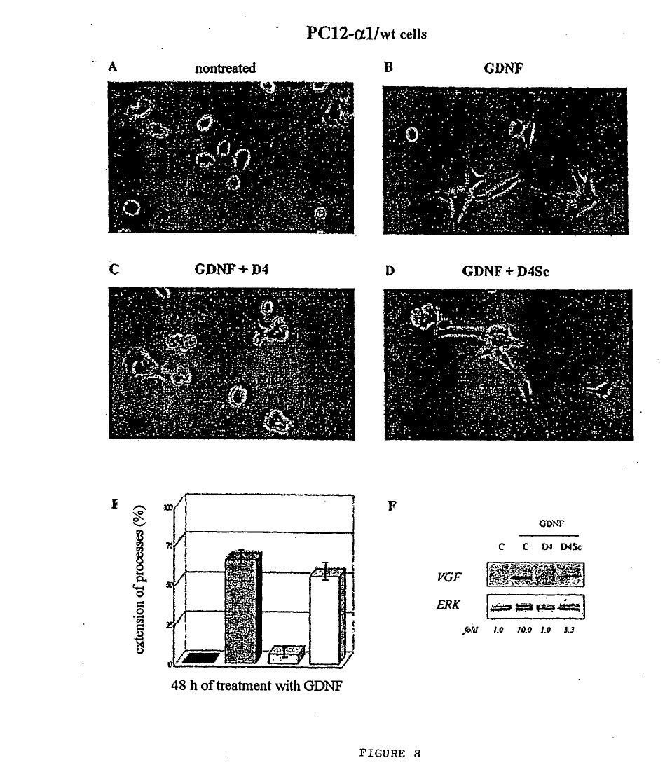

FIG. 9: the D4 aptamer modifies the morphology of the transformed NIH/MEN 2A cells. The cell lines indicated are seeded at equal density onto culture plates comprising 12 wells. One day after seeding, 3 μM of D4 or of destructured D4 are added to the medium and the cells are maintained in culture for 72 hours, adding 3 μM of each aptamer every 24 hours. Given the half-life of the aptamer in 15% serum, this protocol ensures the continuous presence of at least 200 nM of aptamer in the medium. The cells are photographed using a phase-contrast microscope.

FIGS. 10 to 15: secondary structure of the following aptamers: formula II (FIG. 10); D4 (FIG. 11); D24 (FIG. 12); D30 (FIG. 13); D12 (FIG. 14) and D71 (FIG. 15).

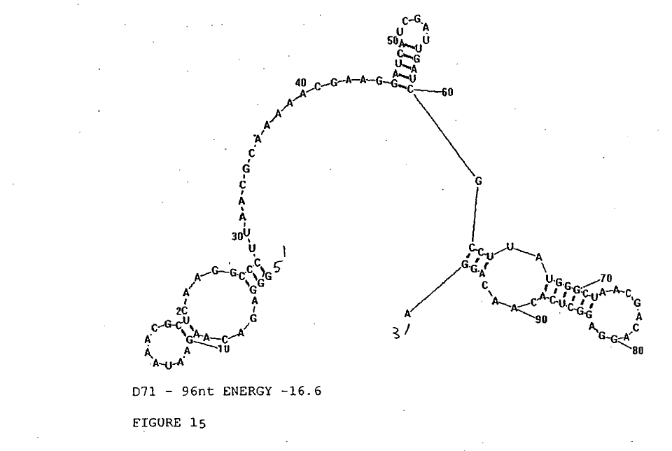

FIG. 16: screening of aptamers which interact with RET on PC12 MEN 2A cells, by competitive binding with the D4 aptamer. The D4 aptamer is radiolabeled with ³²P and incubated at 50 nM with monolayers of PC12 MEN 2A cells in the presence of 400 nM of various aptamers. After several washes, the amount of D4 aptamer bound is quantified. The background noise is taken into account by subtracting, for each point obtained, the value obtained with a destructured D4 aptamer (D4Sc) having a scrambled sequence (i.e. containing the same nucleotides, but in a different order).

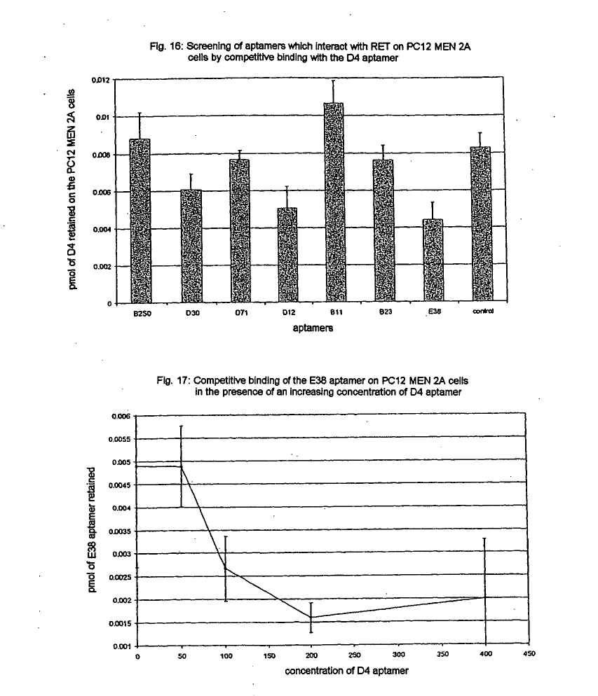

FIG. 17: competitive binding of the E38 aptamer on PC12 MEN 2A cells in the presence of a range of D4 aptamer. The E38 aptamer is radiolabeled with ³²P and incubated at 100 nM with monolayers of PC12 MEN 2A cells in the presence of an increasing concentration of D4 aptamer. After several washes, the amount of E38 aptamer bound is quantified. The background noise is taken into account by subtracting, for each point obtained, the value obtained with a destructured D4 aptamer (D4Sc) having a scrambled sequence (i.e. containing the same nucleotides, but in a different order).

It should be clearly understood, however, that these examples are given only by way of illustration of the subject matter of the invention, of which they in no way constitute a limitation.

### EXAMPLE 1

### Preparation of a Combinatorial Library of 2′-F-Py RNAs

In order to obtain the 2′-F-Py RNA, it is necessary to carry out an in vitro transcription from a double-stranded DNA template obtained according to 3 methods:

### 1. Before Selection, by PCR Amplification of the DNA Sequence:

B2S0:
5′TCCTGTTGTGAGCCTCCTGTCGTT-N-TTGAGCGTTTATTCTTGTCTCCC3′

where N represents a random sequence of 50 nucleotides 1st PCR cycle:

| | |
|---|---|
| * Hybridization | |
| (SEQ ID No:16) | |
| 5′TCCTGTTGTGAGCCTCCTGTCGTT-N-TTGAGCGTTTATTCTTGTCTCCC3′ | (B2S0) |
| (primer P10) 3′AACTCGCAAATAAGAACAGAGGGATATCACTCAGCATAAT5′ | |
| * Elongation | |
| 5′TCCTGTTGTGAGCCTCCTGTCGTT-N-TTGAGCGTTTATTCTTGTCTCCCTATAGTGAGTCGTATTA3′ | |
| 3′AGGACAACACTCGGAGGACAGCAA-M-AACTCGCAAATAAGAACAGAGGGATATCACTCAGCATAAT5′, | |

where the text in bold represents the polymerized sequence and M represents the sequence complementary to N.

### 2nd PCR Cycle:

* Denaturation
5′TCCTGTTGTGAGCCTCCTGTCGTT-N-TTGAGCGTTTATTCTTGTCTCCCTATAGTGAGTCGTATTA3′
3′AGGACAACACTCGGAGGACAGCAA-M-AACTCGCAAATAAGAACAGAGGGATATCACTCAGCATAAT5′
* Hybridization
5′TCCTGTTGTGAGCCTCCTGTCGTT-N-TTGAGCGTTTATTCTTGTCTCCCTATAGTGAGTCGTATTA3′
3′AACTCGCAAATAAGAACAGAGGGATATCACTCAGCATAAT5′
(primer P10: SEQ ID NO:16)
5′TCCTGTTGTGAGCCTCCTGTCGTT3′ (primer P30 SEQ ID NO:17)
3′AGGACAACACTCGGAGGACAGCAA-M-AACTCGCAAATAAGAACAGAGGGATATCACTCAGCATAAT5′
* Elongation
5′TCCTGTTGTGAGCCTCCTGTCGTT-N-TTGAGCGTTTATTCTTGTCTCCCTATAGTGAGTCGTATTA3′
3′AGGACAACACTCGGAGGACAGCAA-M-AACTCGCAAATAAGAACAGAGGGATATCACTCAGCATAAT5′
5′TCCTGTTGTGAGCCTCCTGTCGTT-N-TTGAGCGTTTATTCTTGTCTCCCTATAGTGAGTCGTATTA3′
3′AGGACAACACTCGGAGGACAGCAA-M-AACTCGCAAATAAGAACAGAGGGATATCACTCAGCATAAT5′

the text in bold representing the polymerized sequence and M represents the sequence complementary to N.

This second PCR cycle is repeated 15 to 30 times in order to obtain a double-stranded DNA which will be transcribed, in vitro, into 2′-F-Py RNA.

### 2. During Selection, by RT-PCR Amplification of the Selected 2′-F-Py RNAs, of Formula R₁-R-R₂, as Defined Above:

5′GGGAGACAAGAAUAAACGCUCAA-R-AACGACAGGAGGCUCACAACAGGA3′,

where R represents the sequence of the 2′-F-Py RNAs selected.

### Reverse Transcription (RT):

* Hybridization
5′GGGAGACAAGAAUAAACGCUCAA-R-AACGACAGGAGGCUCACAACAGGA3′ (2′-F-PyRNA)
3′ TTGCTGTCCTCCGAGTGTTGTCCT5′ (primer P30)
* Elongation
5′GGGAGACAAGAAUAAACGCUCAA-R-AACGACAGGAGGCUCACAACAGGA3′
3′CCCTCTGTTCTTATTTGCGAGTT-S-TTGCTGTCCTCCGAGTGTTGTCCT5′,

where the text in bold represents the polymerized sequence and S represents the sequence complementary to R.

### 1st PCR Cycle:

* Denaturation
3′CCCTCTGTTCTTATTTGCGAGTT-S-TTGCTGTCCTCCGAGTGTTGTCCT5′
(cDNA of the 2′-F-Py RNA)
* Hybridization
5′TAATACGACTCACTATAGGGAGACAAGAATAAACGCTCAA3′ (primer P10)
3′CCCTCTGTTCTTATTTGCGAGTT-S-TTGCTGTCCTCCGAGTGTTGTCCT5′
* Elongation
5′TAATACGACTCACTATAGGGAGACAAGAATAAACGCTCAA-R-AACGACAGGAGGCTCACAACAGGA3′
3′ATTATGCTGAGTGATATCCCTCTGTTCTTATTTGCGAGTT-S-TTGCTGTCCTCCGAGTGTTGTCCT5′

where the text in bold represents the polymerized sequence and S represents the sequence complementary to R.

### 2nd PCR Cycle:

* Denaturation
5′TAATACGACTCACTATAGGGAGACAAGAATAAACGCTCAA-R-AACGACAGGAGGCTCACAACAGGA3′
3′ATTATGCTGAGTGATATCCCTCTGTTCTTATTTGCGAGTT-S-TTGCTGTCCTCCGAGTGTTGTCCT5′
* Hybridation
5′TAATACGACTCACTATAGGGAGACAAGAATAAACGCTCAA-R-AACGACAGGAGGCTCACAACAGGA3′
(primer P30) 3′TTGCTGTCCTCCGAGTGTTGTCCT5′
5′TAATACGACTCACTATAGGGAGACAAGAATAAACGCTCAA3′ (primer P10)
3′ATTATGCTGAGTGATATCCCTCTGTTCTTATTTGCGAGTT-S-TTGCTGTCCTCCGAGTGTTGTCCT5′
* Elongation
5′TAATACGACTCACTATAGGGAGACAAGAATAAACGCTCAA-R-AACGACAGGAGGCTCACAACAGGA3′
3′ATTATGCTGAGTGATATCCCTCTGTTCTTATTTGCGAGTT-S-TTGCTGTCCTCCGAGTGTTGTCCT5′
5′TAATACGACTCACTATAGGGAGACAAGAATAAACGCTCAA-R-AACGACAGGAGGCTCACAACAGGA3′
3′ATTATGCTGAGTGATATCCCTCTGTTCTTATTTGCGAGTT-S-TTGCTGTCCTCCGAGTGTTGTCCT5′

where the text in bold represents the polymerized sequence and S represents the sequence complementary to R.

This second PCR cycle is repeated 15 to 30 times in order to obtain a double-stranded DNA which will be transcribed, in vitro, into 2′-F-Py RNA.

3. After Selection, by PCR Amplification of the Aptamers from Plasmid in which they have been Cloned

The plasmids contain the sequence:

5′TAATACGACTCACTATAGGGAGACAAGAATAAACGCTCAA-R-AACGACAGGAGGCTCACAACAGGA3′
3′ATTATGCTGAGTGATATCCCTCTGTTCTTATTTGCGAGTT-S-TTGCTGTCCTCCGAGTGTTGTCCT5′

where R represents the DNA sequence specific for the aptamer and S the sequence complementary to R.

### 1st PCR Cycle:

* Denaturation
5′TAATACGACTCACTATAGGGAGACAAGAATAAACGCTCAA-R-AACGACAGGAGGCTCACAACAGGA3′
3′ATTATGCTGAGTGATATCCCTCTGTTCTTATTTGCGAGTT-S-TTGCTGTCCTCCGAGTGTTGTCCT5′
* Hybridization
5′TAATACGACTCACTATAGGGAGACAAGAATAAACGCTCAA-R-AACGACAGGAGGCTCACAACAGGA3′
(primer P30) 3′TTGCTGTCCTCCGAGTGTTGTCCT5′
5′TAATACGACTCACTATAGGGAGACAAGAATAAACGCTCAA3′ (primer P10)
3′ATTATGCTGAGTGATATCCCTCTGTTCTTATTTGCGAGTT-S-TTGCTGTCCTCCGAGTGTTGTCCT5′
* Elongation
5′TAATACGACTCACTATAGGGAGACAAGAATAAACGCTCAA-R-AACGACAGGAGGCTCACAACAGGA3′
3′ATTATGCTGAGTGATATCCCTCTGTTCTTATTTGCGAGTT-S-TTGCTGTCCTCCGAGTGTTGTCCT5′
5′TAATACGACTCACTATAGGGAGACAAGAATAAACGCTCAA-R-AACGACAGGAGGCTCACAACAGGA3′
3′ATTATGCTGAGTGATATCCCTCTGTTCTTATTTGCGAGTT-S-TTGCTGTCCTCCGAGTGTTGTCCT5′

where the text in bold represents the polymerized sequence and S represents the sequence complementary to R.

This PCR cycle is repeated 15 to 30 times in order to obtain a double-stranded DNA which will be transcribed, in vitro, into 2′-F-Py RNA.

### In Vitro Transcription:

One of the two strands of the PCR-amplified DNA serves as a template for the in vitro transcription of the double-stranded 2′-F-Py RNAs. The sequence underlined corresponds to the region of the T7 phage RNA polymerase promoter.

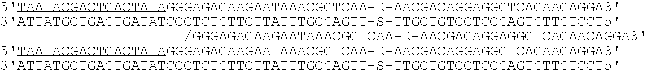

In the 2′-F-Py RNAs, the sequence complementary to the primer P30 is at the 3′ end:

| | |
|---|---|
| 5′AACGACAGGAGGCUCACAACAGGA3′ | (R2 = SEQ ID NO:2) |

and a part of sequence identical to the primer P10 is at the 5′ end:

| | |
|---|---|
| 5′GGGAGACAAGAAUAAACGCUCAA3′ | (R1 = SEQ ID NO:1) |

### EXAMPLE 2

### Materials and Methods

Cell Culture and Immunoblotting Analysis

The conditions for growth of the PC12 cells and of the derived cell lines were described by D'Alessio A. et al. (Endocrinology 2003; 144, 10, 4298-4305).

The NIH/MEN2A and NIH/MEN2B cells are obtained from NIH 3T3 cells stably transfected with expression vectors for Ret^{C634Y} and Ret^{M918T}. In order to evaluate the effects of the aptamers according to the invention on the Ret activity, cells (160 000 cells/3.5 cm of plate) were serum-deprived for 2 hours, and then treated with the amount indicated in the figures (200 nM) of aptamer or of a pool of primary RNA after a short denaturation-renaturation step.

When it is indicated, 100 ng/ml of 2.5 S NGF (Nerve Growth Factor, Upstate Biotechnology Inc., Lake Placid), 50 ng/ml of GDNF (Promega) or 1.6 nM of GFRα1-FC chimera (R&D Systems Ltd., UK) are added to the culture medium.

The cell extracts and the immunoblotting analysis are carried out as described in Cerchia L. et al. (Biochem. J. 2003, 372, 897-903).

The primary antibodies used are as follows: anti-Ret antibody (C-19), anti-VGF antibody (R-15), anti-ERKI antibody (C-16) (Santa Cruz Biotechnology Inc., Santa Cruz Calif.), anti-Ret (Tyr phosphorylated) antibody (Cell Signaling), anti-phospho44/42 MAP kinase monoclonal antibodies (E10) (Cell Signaling). For the immunoblots illustrated in the figures, the statistical analysis was carried out on at least four independent experiments.

Cell Process Extension Assay

PC12-α1/wt cells are seeded at equal density onto culture plates comprising 12 wells. In order to evaluate the effects of the D4 aptamer on cell differentiation, the cells are pretreated for 6 hours with 400 nM of D4 aptamer or of destructured D4 aptamer, and then incubated with 50 ng/ml of GDNF and the appropriate aptamers at a final concentration of 3 μm. After stimulation with GDNF for 24 hours, 3 μM of D4 aptamer or of destructured D4 aptamer are again added to the cells and the stimulation is pursued up to 48 hours. At least 15 random fields are photographed 24 hours and 48 hours after the stimulation with GDNF, using a phase-contrast microscope, and 50 cells per frame are counted; the presence or the absence of process extension is recorded. It is considered that process extension exists when an extension process having a diameter more than double the diameter of the cell body is observed.

SELEX Ex Vivo

The SELEX cycle is carried out essentially as described previously (Tuerk C et al., Science 1990, 249, 4968, 505-510; Ellington A D et al., Nature, 1990, 346, 6287, 818-22). The transcription is carried out in the presence of 1 mM of 2′-F-pyrimidines and of a mutant form of T7 RNA polymerase (T7^{Y639F}) (Padilla, R et al., Nucleic Acids Res, 1999, 27, 6, 1561-1563), in order to increase the yields. The 2′-F-Py RNAs are used because of their resistance to degradation by serum nucleases.

The complexity of the initial sample is approximately 10¹⁴ different sequences. The 2′-F-Py RNA library (1-5 nmol) containing 50 nucleotides of random sequences is heated at 85° C. for 5 min in 3 ml of RPMI 1640, rapidly cooled in ice, for 2 min, and then reheated up to 37° C., before incubation thereof with the cells.

Two counterselection steps are carried out at each cycle.

In order to avoid the selection of aptamers which recognize the cell surface non-specifically, the combinatorial library of initial RNAs is first incubated for 30 minutes at 37° C. with 5×10⁶ PC12 cells (reference ECACC No. 88022) and the unbound sequences are recovered by centrifugation. The latter sequences are then incubated with 5×10⁶ adherent PC12 MEN B2 cells, expressing a Ret receptor mutated in the intracellular domain (Ret^{M918T}), and the unbound sequences are recovered for the selection phase. This step makes it possible to select sequences which specifically recognize the PC12 MEN 2A cells expressing the Ret receptor mutated in the extracellular domain (Ret9^{C634Y}).

The recovered sequences are incubated with 5×10⁶ PC12 MEN 2A cells for 30 min at 37° C. in the presence of non-specific competitive RNA (total yeast RNA, Sigma) and recovered after several washes by total extraction of the RNA (Trizol, Sigma).

After an amplification by RT-PCR using the following pair of primers:

sense primer: 5′ TAATACGACTCACTATAGGGAGACAAGAATAAACGCTCAA 3′ (SEQ ID NO: 16)

| | |
|---|---|
| antisense primer: | |
| 5′ TCCTGTTGTGAGCCTCCTGTCGTT 3′ | (SEQ ID NO:17) |

and an in vitro transcription with mutant T7 polymerase according to the conditions described in Padilla, R et al. (N.A.R., 1999, mentioned above), using a modified buffer (40 mM Tris, pH 7.5; 6 mM MgCl₂; 4 mM NaCl; 2 mM spermidine; 10 mM DTT), the process is repeated. All the incubations with the cells are carried out at 37° C. in RPMI 1640 culture medium, in order to be as close to physiological conditions as possible.

During the selection process, the selection pressure is increased by increasing the number of washes and the amount of non-specific competitive RNA, and decreasing the incubation time and the number of cells exposed to the aptamers, as illustrated in Table II below.

TABLE II
Conditions used for the successive selection cycles

| | Number | Nanomoles | Incubation | Concentration | Incubation | Number | |
|---|---|---|---|---|---|---|---|
| Cycle | (millions) | of 2′-F- | volume | (nM) of 2′-F- | period | of | Competitor |
| No. | of cells | Py RNA | (ml) | Py RNA | (min) | washes | added |
| S1 | 5 | 1 | 3 | 333 | 30 | 1 | 0 |
| S2 | 5 | 3 | 3 | 1000 | 30 | 1 | 0 |
| S3 | 5 | 3 | 3 | 1000 | 30 | 2 | 0 |
| S4 | 5 | 0.5 | 3 | 167 | 30 | 2 | 0 |
| S5 | 5 | 1.5 | 3 | 500 | 15 | 3 | 0 |
| S6 | 5 | 4 | 3 | 1333 | 15 | 4 | 0 |
| S7 | 5 | 1.3 | 3 | 433 | 15 | 3 | 0 |
| S8 | 5 | 2.3 | 3 | 767 | 15 | 3 | 0 |
| S9 | 5 | 2.5 | 3 | 833 | 15 | 3 | 0 |
| S10 | 5 | 10 | 3 | 3333 | 15 | 3 | 150 μg tRNA |
| S11 | 5 | 5.8 | 3 | 1933 | 15 | 3 | 150 μg tRNA |
| S12 | 5 | 2 | 3 | 667 | 15 | 4 | 150 μg tRNA |
| S13 | 2 | 1 | 3 | 333 | 15 | 5 | 150 μg tRNA |
| S14 | 2 | 1 | 3 | 333 | 15 | 5 | 150 μg tRNA |
| S15 | 2 | 1 | 3 | 333 | 15 | 5 | 150 μg tRNA |

In order to follow the evolution of the pool, the appearance of restriction sites containing 4 bases in the population was analyzed by RFLP and reveals the emergence of sequences selected during the SELEX process, which correspond to specific restriction sites (Bartel D P et al., Science, 1993, 261, 5127, 1411-8).

After fifteen selection cycles, the sequences are cloned using the TOPO-TA cloning kit (Invitrogen) and analyzed by sequencing.

Binding Assays

The binding of the various aptamers (or of the initial pool, as control) to the PC12 MEN 2A cells is carried out in 24-well plates (experiments carried out in triplicate), with 5′-³²P-labeled RNA. 10⁵ cells per well are incubated with various concentrations of aptamers in 200 μl of RPMI for 10 min at 37° C. in the presence of 100 μg/ml of polyinosine, as non-specific competitor. After several washes, the bound sequences are recovered in 350 μl of 0.6% SDS and the amount of radioactivity recovered is related to the number of cells by measuring the protein content in each well.

The dissociation constants (Kd) and the number of targets (Tₘₐₓ) for each aptamer are determined:
by Scatchard analysis according to the following equation:

[bound aptamer]/[aptamer]=−(1/Kd)×[bound aptamer]+[Tmax]/Kd
or by analysis according to the Lineweaver-Burk method, according to the following equation:

1/[bound aptamer]=Kd/([Tmax]×[aptamer])+1/[Tmax].

The binding of the individual sequences to the various cell lines is carried out under the same conditions, but at a single concentration of 50 nM.

### EXAMPLE 3

### Results

SELEX Ex Vivo: Selection of Aptamers

In order to identify the aptamers which recognize the Ret receptor, a modified SELEX protocol was carried out, as specified in example 1:
the method is carried out on intact cells, i.e. a cell line derived from PC12 cells (PC12 MEN 2A), expressing the human mutant receptor Ret^{C634Y};
2′-F-pyrimidine RNAs are used to increase the nuclease-resistance of the aptamers present in the biological fluids;
two counterselection steps are carried out at each cycle: (1) on PC12 cells which do not express the human Ret receptor and (2) on PC12 MEN 2B cells which express an allele mutated in the intracellular tyrosine kinase domain of the Ret receptor (Ret^{M918T}), so as to optimize the selection of aptamers specific for the Ret receptor.

The context used made it possible to select aptamers capable of targeting surface epitopes specific for PC12 MEN 2A cells and also aptamers capable of recognizing the extracellular portion of the Ret receptor.

This technique has the advantage of avoiding the use of recombinant Ret receptor.

69 clones were obtained and sequenced, from the pool of sequences obtained at the 15th round and bound to the PC12 MEN 2A cells in a saturable manner with a Kd of approximately 100 nM. The results are illustrated in Table III below.

TABLE III

Results of the selection (the sequences are given without the fixed
regions R1 (SEQ ID NO:1) and R2 (SEQ ID NO:2) which border them and
allow RT-PCR amplification)

| | | | | |
|---|---|---|---|---|
| | | binding at | | |
| Sequence code | | 100 nM | Kd on | number of |
| (number of | | on PC12 | PC12 MEN | targets/cell |
| clones) | Sequence (R) | MEN 2A | 2A (nM) | (× 1000) |

| | | | | |
|---|---|---|---|---|
| D14(23) | GGCCATAGCGCACCACCAAGAGCAAAT | | | |
| | CCCTAAGCGCGACTCGAGTGAGC | − | | |
| D12(21) | GGGCUUCAUAAGCUACACCGGCCAAC | | 67 ± 7 | 102 ± 25 |
| | GCAGAAAUGCCUUAAGCCCGAGUU | + | | |
| D30(7) | AGGCGAGCCCGACCACGTCAGTATGCT | | 74 ± 25 | 212 ± 27 |
| | AGACAACAACGCCCGCGTGGTAC | + | | |
| D71(5) | GGCCCUUAACGCAAAAACGAAGGAUCA | | 63 ± 7 | 248 ± 42 |
| | UCGAUUGAUCGCGUUAUGGGCU | + | | |
| D42(4) | GACCCGUAUGAAGGUGGCGCAGGACA | | | |
| | CGACCGUCUGCAAUGAGCGAGC | − | | |
| D20(2) | GGGCCAATCGAAGCCGGTAATTCCCAA | | | |
| | ACTAACGTGCAAACTGCACCCGC | − | | |
| D76 | GGCTTACACGGAGAAACAAGAGAGCGG | | | |
| | CCCAAACTTGATTGACAGTGGCC | − | | |
| D60 | CCGACCTGTACAGCAGTTAGTTACACG | | | |
| | TTTGAAACAACCGGCGTTCGAGC | − | | |
| D32 | CCCCGCTTTTTGACGTGATCGAACGCG | | | |
| | TATCAGTAACGTCAGCAGTCGAGC | − | | |
| D33 | CAAAGCGTGTATTCTCGTGAGGCGACC | | | |
| | ATCGTTGCGAACATCCCCGGAACG | − | | |
| D87 | CCGCGGTCTGTGGGACCCTTCAGGATG | | | |
| | AAGCGGCAACCCATGCGGGCC | − | | |
| D24 | GCGGTATGTAGGGAATAGCACTTTTTTT | | 32 ± 5 | 102 ± 58 |
| | GCGTATACCTACACCGCAGCG | + | | |
| D4 | GCGCGGGAATAGTATGGAAGGATACGT | | 35 ± 3 | 110 ± 47 |
| | ATACCGTGCAATCCAGGGCAACG | + | | |

Two sequences (D14 and D12) dominate the selection and constitute more than 50% of the clones. Four other sequences are less abundant (25% of the clones) and the others are present only once (seven sequences).

Due to the complexity of the target (live whole cells), no similarity was observed between the various aptamer sequences, except with regard to clones D24 and D4, which share certain motifs and a common structure (see FIG. 5A, formula II and FIGS. 11 and 12).

The ability of each aptamer to bind to PC12 MEN 2A cells was tested.

All the sequences found more than once were thus tested, along with sequences present in a lesser amount (including D4 and D24). Despite its abundance, D14 does not bind the PC12 MEN 2A cells significantly above the background noise.

Other sequences bind the PC12 MEN 2A cells, with a Kd of between 30 and 70 nM.

Most of the aptamers do not bind the parental PC12 cells, rat bladder carcinoma cells (NBTII) and human HeLa cells.

Action of the D4 Aptamer on the Ret Receptor

The activation of the normal Ret receptor in normal cells (Cn) occurs via interaction with the GRF-alpha coreceptor for several trophic factoprs, the most widespread of which is GDNF (Glial Derived Growth Factor) (FIG. 4). In cells expressing the normal form of Ret, the cascade is activated only in the presence of GDNF. Inhibition of the phosphorylation of Ret by GDNF in these cells in the presence of D4 is proof that D4 interacts with the Ret signaling pathway, which confirms the absence of activity of D4 on the activation by another trophic factor, NGF, the activity of which on Erk is not mediated by Ret. The fact that D4 does not have any activity, either, on the activation of Ret and Erk in MEN 2B cells in which the mutation is intracellular suggests that the interaction between D4 and Ret involves the extracellular portion of Ret, and may be at the dimerization site. However, this is not proved.

Very notably, the activity of D4 leads to a reversion of the transformed phenotype on cells in which Ret is constitutively activated. These cells in culture take on a “neuronal-like” morphotype with axonal extensions. D4, but not D4Sc, which is a destructured. D4, having the same chemical composition but without ordering of its sequence in an active structure, induces a very significant reduction in the number of axonal extensions and brings the cell phenotype back to a nonactivated-cell phenotype, both in a neuroendocrine line (PC 12) and in a fibroblast line (NIH 3T3).

The mutant Ret^{C634Y} receptor, expressed by the PC12 MEN 2A cells, forms homodimers at the cell surface, which leads to constitutive activation of its tyrosine kinase activity (Santoro M et al., Science, 1995, 20, 267, 5196, 381-383) and induces several downstream signaling cascades, including the activation of Erk kinase (Colucci-D'Amato et al., J. Biol. Chem., 2000, 275, 19306-19314; Jhiang S M, Oncogene, 2000, 19, 5590-5597). Using PC12 cells expressing the Ret^{C634Y} oncogene as in vitro cell system, the ability of each aptamer to inhibit the autophosphorylation of the Ret^{C634Y} receptor and the downstream signaling dependent on the receptor (FIG. 6) is evaluated.

The PC12 MEN 2A cells are incubated overnight with one of the following aptamers: D4 (SEQ ID NO:3), D12 (SEQ ID NO:4), D30 (SEQ ID NO:8) and D71 (SEQ ID NO:14), at a final concentration of 150 nM. The cell lysates are analyzed by immunoblotting with anti(Tyr-phosphorylated) Ret antibodies (FIG. 6B) or anti-phospho-Erk antibodies (FIG. 6B). As has already been demonstrated (Colucci et al., mentioned above), the levels of phosphorylated Ret receptor and of phosphorylated Erk protein are constitutively high in nontreated PC12 MEN 2A cells, due to the presence of the active Ret^{C634Y} allele. Some of the aptamers tested inhibit the autophosphorylation of the Ret^{C634Y} receptor and the phosphorylation of the Erk protein which results therefrom, in comparison with the starting combinatorial library and with other aptamers (FIG. 6A). Using these experimental conditions, the D4 aptamer is found to be the most effective inhibitor and was therefore used for the following studies.

Under these conditions, a dose-response experiment (FIG. 6B, left) reveals that a concentration of 200 nM of D4 aptamer is sufficient to inhibit the autophosphorylation of the Ret^{C634Y} receptor by up to 70% and to very substantially reduce the phosphorylation of the Erk protein. Treatment of the cells for one hour with 200 nM of D4 aptamer is sufficient to significantly inhibit the autophosphorylation of the Ret^{C634Y} receptor and to completely abolish the phosphorylation of the Erk protein (FIG. 6B, right).

In all the experiments, the inhibition of the phosphorylation of the Erk protein is more rapid and more quantitative than the inhibition of the phosphorylation of the Ret^{C634Y} receptor, doubtless due to different sensitivities of the two processes for modifying the tyrosine kinase activity of the Ret receptor.

The predicted secondary structure of the D4 aptamer is illustrated in FIG. 5A, as is that of the D24 aptamer. Comparison of the two structures suggests that the cell binding is not dependent on the sequence of the tail or of the apical loop. In fact, if the apical loop is replaced with an extra-stable loop comprising four nucleotides (UUGC) or by deleting the nucleotides represented by R₄ and R₅, as defined above, no significant difference is observed in binding to the PC12 MEN 2A cells. However, it is the complete D4 aptamer which is the product most active in inhibiting the signaling pathway induced by the Ret^{C634Y} receptor. A 2′-F-Py RNA of identical composition but with a destructured sequence (D4Sc) is ineffective both in terms of binding and in terms of inhibition.

The D4 aptamer recognizes the PC12 MEN 2A cells with an estimated Kd of 35 nM (FIG. 5B); furthermore, it recognizes neither the parental PC12 cells, nor rat NBTII cells, nor human HeLa cells, which do not express the Ret receptor.

Insofar as the D4 aptamer was selected on cells expressing the mutant Ret^{C634Y} receptor, an attempt was made to determine whether the D4 aptamer could also inhibit the wild-type Ret receptor. With this aim, a cell line derived from PC12 cells expressing the wild-type Ret receptor (PC12/wt) was used. The cells were stimulated with a mixture containing GDNF and soluble GFRα1 and were treated with the D4 aptamer or with the starting combinatorial library as negative control. As illustrated in FIG. 7A, only the D4 aptamer (and not the control combinatorial RNA library) inhibits, substantially, the phosphorylation of the Ret receptor, induced by GDNF (FIG. 7A), and of the Erk protein (FIG. 7B). A similar inhibitory effect was observed with the PC12-α1/wt cells, a cell line derived from PC12 cells which stably expresses both the human Ret receptor and GFRα1. It is important to note that the D4 aptamer is inactive on the signaling pathway induced by NGF on the tyrosine kinase receptor TrkA. After stimulation of the cells with NGF, the treatment with the D4 aptamer does not modify the amount of phospho-Erk, which indicates that the D4-aptamer-induced inhibition of the phosphorylation of the Erk protein is specific for the GDNF-stimulated intracellular signaling pathway of the Ret receptor (FIG. 7).

Although the D4 aptamer binds the PC12 MEN 2B cells, treatment of these cells with 200 nM of D4 for one hour (FIG. 7B) does not interfere with the signaling pathway induced by the monomeric Ret^{M918T} receptor. This confirms that the inhibition of the phosphorylation of the Erk protein by the D4 aptamer is specific for the form activated by dimerization of the Ret receptor. The kinase and biological activities of the Ret^{M918T} receptor, although constitutive, respond to a stimulation with GDNF in the presence of GFRα1 (Carlomagno F et al., Endocrinology, 1998, 139, 8, 3613-3619). However, in accordance with the inhibition of the activity of the wild-type Ret receptor (Ret wt) by the D4 aptamer, treatment of the PC12 MEN 2B cells with the D4 aptamer abolishes the overstimulation of the phosphorylation of the Ret receptor and of the Erk protein, dependent on GDNF. These experiments show that the D4 aptamer inhibits the activities of both the Ret receptor and the Erk protein by direct action on the Ret receptor and not by action on other cell targets, for example tyrosine phosphatase.

Biological Effects of the D4 Aptamer on Ret Receptor-Dependent Cell Differentiation and Transformation

The observation that the inhibition occurs in all cases where dimerization of the wild-type (wt) or mutant receptor is necessary for the signaling pathway also indicates that the dimerization is the target of the action of the D4 aptamer.

With this aim, the axonal extension (or neural crest) was measured as the reflection of the differentiation in PC12-α1/wt cells after stimulation with GDNF (see example 1).

The cells are treated with 50 ng/ml of GDNF and the percentage of cells containing axonal extensions is determined 24 and 48 hours after the treatment as specified in example 1. As illustrated in FIG. 8, the cells exhibit axonal extension processes in response to exposure to GDNF for two days (FIG. 8B), compared with the nonstimulated control cells (FIG. 8A). The treatment of the cells with the D4 aptamer (FIG. 8C), but not with the destructured D4 aptamer (D4Sc) used as control (FIG. 8D), significantly inhibits the number and the length of the axons, perhaps by preventing the formation of a functional complex between the Ret receptor and GDNF/GFRα1. In order to biochemically evaluate the differentiation, the VGF levels in cell extracts were determined after 48 hours of treatment. Vgf is an early gene which is rapidly induced by NGF and GDNF in PC12 cells (Salton S R, Mt Sinai J Med., 2003, 70, 2, 93-100). It is observed (FIG. 8F) that, in the cells treated with GDNF, the expression of VGF is stimulated and in accordance with the phenotypic effects reported above, the treatment with the D4 aptamer, but not the treatment with the destructured D4 aptamer, maintains VGF levels close to basal levels.

After expression of the Ret^{C634Y} receptor or of the Ret^{M918T} receptor, NIH 3T3 cells are transformed and exhibit considerable changes in their morphology (Santoro et al., Science, 1995, mentioned above). NIH/MEN 2A cells and NIH/MEN 2B cells, which stably express the mutant Ret receptors, are treated with the D4 aptamer for 72 hours and the morphological modifications induced by this aptamer are analyzed. As illustrated in FIG. 9, the NIH/MEN 2A cells and the NIH/MEN 2B cells have a spindle shape, long protrusions and a highly refringent appearance (FIGS. 9B and 9E, respectively). The NIH/MEN 2A cells treated with the D4 aptamer return to a polygonal and flat morphology similar to that of the parental NIH 3T3 cells (FIG. 9C), whereas no morphological change is observed in the NIH/MEN 2B cells (FIG. 9F) or in the NIH-Ras cells. This is in agreement with the previous results which show that the signaling pathway induced by the Ret^{C634Y} receptor, but not that induced by the Ret^{M918T} receptor is inhibited by the D4 aptamer.

Moreover, the treatment with the destructured D4 aptamer has no effect on the various cell lines (FIG. 9D). Consequently, treatment of the NIH/MEN 2A cells for 72 hours with the D4 aptamer inhibits the extent of the effects of the activation of the Erk protein by phosphorylation both of the Ret^{C634Y} receptor and of the Erk protein.

### EXAMPLE 4

### Use of the D4 Aptamer for Screening Products which Interact with the Ret Receptor or Targets which Form a Complex with the Ret Protein on PC12 MEN 2A Cells

In order to validate the use of the D4 aptamer for screening molecules which interact with the Ret receptor or targets which form a complex with said protein on PC12 MEN 2A cells, two approaches were used:
1—The D4 aptamer radiolabeled with ³²P was incubated at 50 nM with monolayers of PC12 MEN 2A cells in the presence of 400 nM of various aptamers selected either on cells or on the isolated recombinant Ret protein. After several washes, the amount of D4 aptamer bound is quantified. By comparing the amount of binding of the D4 aptamer as a function of the molecules used, it can be noted that only the D12 aptamer, and especially the E38 aptamer, the target of which is the Ret receptor, were capable of decreasing the binding of the D4 aptamer to the cells. It can therefore be concluded that these two aptamers bind to a target related to the Ret protein, either directly on said protein, possibly at the same site as the D4 aptamer, or on another target present in a complex with Ret.
2—The E38 aptamer radiolabeled with ³²P was incubated at 100 nM with monolayers of PC12 MEN 2A cells in the presence of an increasing concentration of D4 aptamer. After several washes, the amount of E38 aptamer bound to the cells was quantified. An increasing inhibition of the binding of the E38 aptamer as a function of the concentration of D4 aptamer can be noted. It can therefore be concluded that the E38 aptamer binds to a target related to the Ret protein, either directly on said protein, possibly at the same site as the D4 aptamer, or on another target present in a complex with Ret.

1. A method for identifying ligands or aptamers specific for a membrane receptor protein-tyrosine kinase (RPTK), expressed in an activated or nonactivated form, by cells, using a mixture of nucleic acids, which method comprises at least the following steps:
(a) bringing a mixture of nucleic acids into contact with cells not expressing said receptor protein-tyrosine kinase or expressing it in a nonactivated form (C_{N} cells), said cells having the same cell type as cells expressing the same receptor protein-tyrosine kinase but in an activated form, due to the existence of a mutation in the extracellular domain (C_{Te} cells);
(b) recovering a first subset S1 of nucleic acids which do not bind to the C_{N} cells, in step (a);
(c) bringing said first subset S1 into contact with Cᵢ cells, having the same cell type as the C_{Te} cells, but expressing said receptor protein-tyrosine kinase mutated in its intracellular part, said Cᵢ cells exhibiting a phenotype of the same type as that of the C_{Te} cells;
(d) recovering a second subset S2 of nucleic acids which do not bind to the Cᵢ cells in step (c);
(e) bringing the second subset S2 into contact with the C_{Te} cells;
(f) recovering the nucleic acids which bind to said C_{Te} cells, i.e. those exhibiting a high affinity with respect to the cells expressing said receptor protein-tyrosine kinase mutated in the extracellular domain, after dissociation of the cell-nucleic acid complexes;
(g) amplifying said nucleic acids with high affinity for the cells expressing said receptor protein-tyrosine kinase mutated in the extracellular domain, so as to obtain a mixture of nucleic acids, enriched in nucleic acids having a high affinity for said C_{Te} cells, and
(h) identifying the ligands or aptamers specific for the cells expressing receptor protein-tyrosine kinases (RPTKs) in an activated form, from the mixture obtained in (g).

2. The method as claimed in claim 1, wherein steps (a)-(g) are repeated using the mixtures enriched in ligands or aptamers from the preceding cycle, until at least one aptamer is obtained, the affinity of said aptamer, defined by its dissociation constant (Kd), can be measured and is suitable for pharmaceutical use.

3. The method of claim 1 wherein the starting nucleic acid combinatorial library contains at least 10² nucleic acids.

4. The method of claim 3, wherein said starting nucleic acid combinatorial library consists of nucleic acids comprising random sequences each containing between 10 and 1000 nucleotides.

5. The method of claim 1 wherein the identification of the ligands or aptamers specific for the C_{Te} cells according to step (h) comprises an evaluation of the biological activity of said aptamers on said C_{Te} cells.

6. The method of claim 5, wherein said biological activity which is evaluated comprises the following:
(a) inhibition or activation of the auto-phosphorylation of the RPTK,
(b) inhibition or activation of the kinase activation cascade,
(c) inhibition of the phosphorylation of the normal RPTK of C_{N} cells activated by suitable stimulation, and
(d) reversion of the phenotype associated with activation of the RPTK.

7. An aptamer, wherein said aptamer is specific for cells expressing a receptor protein-tyrosine kinase (RPTK) in an activated or nonactivated form and can be identified by the method for identifying aptamers of claim 1.

8. The aptamer as claimed in claim 7, wherein the receptor protein-tyrosine kinase (RPTK) in an activated or nonactivated form, is selected from the group consisting of: EGFR (Epithelial Growth Factor Receptor), InsulinR (Insulin Receptor), PDGFR (Platelet-derived Growth Factor Receptor), VEGFR (Vascular Endothelial Growth Factor Receptor), FGFR (Fibroblast Growth Factor Receptor), NGFR (Nerve Growth Factor Receptor), HGFR (Hepatocyte Growth Factor Receptor), EPHR (Ephrin Receptor), AXL (Tyro 3 PTK), TIE (Tyrosine Kinase Receptor in endothelial cells), RET (Rearranged During Transfection), ROS(RPTK expressed in certain epithelial cells) and LTK (Leukocyte Tyrosine Kinase).

9. The aptamer as claimed in claim 7 wherein said aptamer recognises a Ret receptor in an activated form.

10. The aptamer as claimed in claim 9, wherein said aptamer can be identified by means of the method comprising:
(a) bringing a mixture of nucleic acids into contact with C_{N} cells not expressing any Ret receptor in an activated form,
(b) recovering a first subset S1 of nucleic acids which do not bind to said C_{N} cells, in step (a),
(c) bringing said first subset S1 into contact with Cᵢ cells expressing a Ret receptor, mutated in its intracellular domain,
(d) recovering a second subset S2 of nucleic acids which do not bind to said Cᵢ cells,
(e) bringing the second subset S2 into contact with C_{Te} cells expressing a Ret receptor activated by mutation in the extracellular domain, which receptor is selected from the group consisting of mutated Ret receptors carrying a mutation on one of the cysteines located in the extracellular domain,
(f) recovering the nucleic acids bound to said C_{Te} cells, exhibiting both a high affinity and a binding specificity for the cells expressing a mutated Ret receptor as defined in step (e),
(g) amplifying said nucleic acids obtained in step (f), to obtain a mixture of nucleic acids, enriched in nucleic acids having a high affinity for the C_{Te} cells,
(h) repeating steps (a)-(g), until at least one aptamer is obtained, the affinity of which for the C_{Te} cells, defined by its dissociation constant (Kd), is measurable and suitable for a pharmacological activity, and
(i) identifying the aptamers specific for the cells expressing a Ret receptor in its activated form, selected from the mixture obtained in (h).

11. The aptamer as claimed in claim 10, wherein
the C_{N} cells are wild-type PC12 cells (reference ECACC No. 88022) or wild-type NIH 3T3 cells (reference ECACC No. 93061524),
the Cᵢ and C_{Te} cells are obtained by introducing an oncogene bearing a mutation, respectively intracellular and extracellular, in C_{N} cells in culture such that the latter express the oncogene.

12. An aptamer, wherein said aptamer can be obtained by the method of claim 1 and is selected from the group consisting of the aptamers of formula (I):
R₁-R-R₂ (I),
in which:
R₁ represents 5′ GGGAGACAAGAAUAAACGCUCAA 3′ (SEQ ID NO:1) or a fragment of 1 to 23 nucleotides of said SEQ ID NO:1;
R₂ represents 5′ AACGACAGGAGGCUCACAACAGGA 3′ (SEQ ID NO:2) or a fragment of 1 to 24 nucleotides of said SEQ ID NO:2, and
R represents a random sequence of 10 to 1000 nucleotides.

13. The aptamer as claimed in claim 12 wherein R is selected from the following sequences:

| | | |
|---|---|---|
| D4 | 5′GCGCGGGAAUAGUAUGGAAGGAUACGUAUACCGUGCAAUCCAGGGCAACG 3′ | (SEQ ID NO:3) |
| D12 | 5′GGGCUUCAUAAGCUACACCGGCCAACGCAGAAAUGCCUUAAGCCCGAGUU 3′ | (SEQ ID NO:4) |
| D14 | 5′GGCCAUAGCGCACCACCAAGAGCAAAUCCCUAAGCGCGACUCGAGUGAGC 3′ | (SEQ ID NO:5) |
| D20 | 5′GGGCCAAUCGAAGCCGGUAAUUCCCAAACUAACGUGCAAACUGCACCCGC 3′ | (SEQ ID NO:6) |
| D24 | 5′GCGGUAUGUAGGGAAUAGCACUUUUUUUGCGUAUACCUACACCGCAGCG 3′ | (SEQ ID NO:7) |
| D30 | 5′AGGCGAGCCCGACCACGUCAGUAUGCUAGACAACAACGCCCGCGUGGUAC 3′ | (SEQ ID NO:8) |
| D32 | 5′CCCCGCUUUUUGACGUGAUCGAACGCGUAUCAGUAACGUCAGCAGUCGAGC 3′ | (SEQ ID NO:9) |
| D33 | 5′CAAAGCGUGUAUUCUCGUGAGCCGACCAUCGUUGCGAACAUCCCCGGAACG 3′ | (SEQ ID NO:10) |
| D42 | 5′GACCCGUAUGAAGGUGGCGCAGGACACGACCGUCUGCAAUGAGCGAGC 3′ | (SEQ ID NO:11) |
| D60 | 5′CCGACCUGUACAGCAGUUAGUUACACGUUUGAAACAACCGGCGUUCGAGC 3′ | (SEQ ID NO:12) |
| D76 | 5′GGCUUACACGGAGAAACAAGAGAGCGGCCCAAACUUGAUUGACAGUGGCC 3′ | (SEQ ID NO:13) |
| D71 | 5′GGCCCUUAACGCAAAAACGAAGGAUCAUCGAUUGAUCGCCUUAUGGGCU 3′ | (SEQ ID NO:14) |
| D87 | 5′CCGCGGUCUGUGGGACCCUUCAGGAUGAAGCGGCAACCCAUGCGGGCC 3′ | (SEQ ID NO:15) |

14. The aptamer as claimed in claim 12 wherein the riboses of the purines bear a hydroxyl function on the carbon in the 2′-position, while the riboses of the pyrimidines bear a fluorine atom on the carbon in the 2′-position.

15. The aptamer as claimed in claim 12 wherein said aptamer has one of the following sequences: SEQ ID NOs:31-33.

16. The aptamer as claimed in claim 12, wherein said aptamer has formula II below:

| | |
|---|---|
| 5′R₄X₆X₅X₄X₃GGAAUAGX₂X₁R₃X′₁X′₂CGUAUACX′₃X′₄X′₅X′₆R₅3′ | (SEQ ID NO: 34) |

(II), wherein:
the riboses of the purines bear an OH group in the 2′-position and the riboses of the pyrimidines bear a fluorine atom in the 2′-position;
R₃ is present or absent and represents an apical bulge comprising:
a linear or branched carbon chain selected from the group consisting of C₆-C₃₀ alkyl groups and C₆-C₃₀ aryl groups;
a polymer selected from the group consisting of PEG and PEI;
functional groups selected from the group consisting of biotin, streptavidin, and peroxidase;
other molecules of interest selected from the group consisting of active ingredients, labeling tags, and chelating agents for radioisotopes;
a natural or modified nucleotide sequence;
X′₁, X′₁, X₂, X′₂, X₃, X′₃, X₄, X′₄, X₅, X′₅, X₆ and X′₆ represent Py or Pu with,
X₁-X′₁ corresponding to C-G, A-U, G-C or U-A
X₂-X′₂ corresponding to C-G, A-U, G-C or U-A
X₃-X′₃ corresponding to C-G, A-U, G-C or U-A
X₄-X′₄ corresponding to C-G, A-U, G-C or U-A
X₅-X′₅ corresponding to C-G, A-U, G-C or U-A
X₆-X′₆ corresponding to C-G, A-U, G-C or U-A
N corresponding to G or C or A or U,
Pu corresponding to G or A, in which the riboses bear an OH group in the 2′-position,
Py corresponds to U or C, in which the riboses bear a fluorine atom in the 2′-position, and
R₄ and R₅ are present or absent and represent:
a natural or modified nucleotide sequence, comprising between 1 and several thousand nucleotides, wherein a part of said nucleotide sequence is selected from the group consisting of the following sequences:
R₄:
5′-R₁-Z₁-3′, with Z₁ = G:
(SEQ ID NO:18)
5′ GGGAGACAAGAAUAAACGCUCAAG 3′,
5′-R₁-Z₁-3′, with Z₁ = GCGGUAU (SEQ ID NO:26):
(SEQ ID NO:19)
5′ GGGAGACAAGAAUAAACGCUCAAGCGGUAU,
and
R₅:
5′-Z₂-R₂-3′, with Z₂ = CAAUCCAGGGCAACG (SEQ ID
NO:27):
(SEQ ID NO:20)
5′CAAUCCAGGGCAACGAACGACAGGAGGCUCACAACAGGA 3′
5′-Z₂-R₂-3′, with Z₂ = ACCGCAGCG (SEQ ID NO:28):
(SEQ ID NO:21)
5′ ACCGCAGCGAACGACAGGAGGCUCACAACAGGA 3′,
(SEQ ID NO:18)
5′ GGGAGACAAGAAUAAACGCUCAAG 3′
(SEQ ID NO:19)
5′ GGGAGACAAGAAUAAACGCUCAAGCGGUAU,
for R₄
and
(SEQ ID NO:20)
5′ CAAUCCAGGGCAACGAACGACAGGAGGCUCACAACAGGA 3′
and
(SEQ ID NO:21)
5′ ACCGCAGCGAACGACAGGAGGCUCACAACAGGA 3′
for R₅;
a linear or branched carbon chain selected from the group consisting of C₆-C₃₀ alkyl groups, C₆-C₃₀ aryl groups
a polymer selected from the group consisting of PEG and PEI;
functional groups selected from the group consisting of biotin, streptavidin and peroxidase;
other molecules of interest, selected from the group consisting of active ingredients, labeling tags, and chelating agents for radioisotopes.

17. The aptamer as claimed in claim 16, wherein R₃ represents 5′ UGGAAGGA 3′ (SEQ ID NO: 29) (loop (1)), R₄ represents SEQ ID NO:18 and R₅ represents SEQ ID NO:20, said aptamer has both properties of binding to a Ret receptor and properties of inhibition of the activity of said receptor.

18. The aptamer as claimed in claim 17, wherein said aptamer has the sequence SEQ ID NO:22.

19. The aptamer as claimed in claim 16, wherein R₃ represents 5′ CUUUUUU 3′ (SEQ ID NO: 30) (loop (2)), 5′ GNPuA 3′ (loop (3)) or 5′ UNCG 3′ (loop (4)), R₄ comprises from 1 to 30 nucleotides selected from SEQ ID NO:19 or from 1 to 24 nucleotides selected from SEQ ID NO:18 and R₅ comprises from 1 to 33 nucleotides of SEQ ID NO:21 or from 1 to 39 nucleotides selected from SEQ ID NO:20, the aptamer of this structure having only properties of binding to a Ret receptor in its activated or nonactivated form.

20. The aptamer as claimed in claim 19, wherein R₃ represents 5′ CUUUUUU 3′ (SEQ ID NO: 30, R₄ represents SEQ ID NO:19 and R₅ represents SEQ ID NO:21.

21. The aptamer as claimed in claim 19 wherein said aptamer has SEQ ID NO:25.

22. The aptamer as claimed in claim 16 wherein said aptamer has the sequence SEQ ID NO:23 and R₃ represents 5′ UGGAAGGA 3′ (SEQ ID NO: 29), R₄ and R₅ are absent, the aptamer of this structure having only properties of binding to a Ret receptor in its activated or nonactivated form.

23. A reagent for diagnosing a tumor, wherein said reagent comprises an aptamer as claimed in claim 12.

24. The reagent as claimed in claim 23, comprising an aptamer of formula II:
5′R₄X₆X₅X₄X₃GGAAUAGX₂X₁R₃X′₁X′₂CGUAUACX′₃X′₄X′₅X′₆R₅3′ (SEQ ID NO: 35 (II), in which R₃, R₄ and R₅ are absent.

25. The reagent as claimed in claim 24, comprising an aptamer of sequence:

| | |
|---|---|
| 5′ GUAGGGAAUAGCACGUAUACCUAC 3′. | (SEQ ID NO:24) |

26. The reagent as claimed in claim 23, comprising an aptamer of formula II, 5′R₄X₆X₅X₄X₃GGAAUAGX₂X₁R₃X′₁X′₂CGUAUACX′₃X′₄X′₅X′₆R₅3′ (SEQ ID NO: 34) (II), in which R₃ represents 5′ CUUUUUU 3′ (SEQ ID NO: 30), said aptamer corresponding to the sequence SEQ ID NO:25.

27. A reagent for diagnosing or detecting a Ret receptor in an activated or nonactivated form, comprising at least one aptamer as claimed in claim 12.

28. A medicament, comprising an aptamer as claimed in claim 7, which has both an ability to bind to an RPTK receptor and an inhibitory action with respect to said receptor in an activated form.

29. A medicament for use in the treatment of a tumor, wherein the medicament comprises an aptamer as claimed in claim 7 which has both an ability to bind to an activated RPTK receptor and an inhibitory action with respect to this receptor.

30. The medicament as claimed in claim 28 comprising an aptamer selected from the group consisting of the aptamers of formula (I):
R₁-R-R₂ (I),
in which:
R₁ represents 5′ GGGAGACAAGAAUAAACGCUCAA 3′ (SEQ ID NO:1) or a fragment of 1 to 23 nucleotides of said SEQ ID NO:1;
R₂ represents 5′ AACGACAGGAGGCUCACAACAGGA 3′ (SEQ ID NO:2) or a fragment of 1 to 24 nucleotides of said SEQ ID NO:2, and
R represents SEQ ID NO. 3.

31. A pharmaceutical composition, comprising an aptamer as claimed in claim 7 which has both an ability to bind to an RPTK receptor and an inhibitory action with respect to said receptor in its activated form.

32. A pharmaceutical composition, comprising:
an aptamer as claimed in claim 7, which has both an ability to bind to an activated RPTK receptor mutated in the extracellular domain, and an inhibitory action with respect to this mutated receptor,
another anticancer molecule, and
at least one pharmaceutically acceptable vehicle.

33. The use of an aptamer which has both an ability to bind to an RPTK receptor and an inhibitory action with respect to this RPTK receptor, for screening products which interact with the RPTK receptor and which may or may not inhibit it comprising:
bringing cells expressing RPTKs in an activated or nonactivated form into contact with the product to be tested,
adding, under suitable conditions, an aptamer of claim 7, before, at the same time as or after the product to be tested,
evaluating the competitive binding between the aptamer and the product to be tested.

34. The use of an aptamer which has both an ability to bind to an activated RPTK receptor mutated at one of the cysteines located in the extracellular domain (codons 609, 611, 618, 620 and 634), and an inhibitory action with respect to this activated RPTK receptor, for screening products which interact with said RPTK receptor, comprising:
bringing cells expressing RPTKs in an activated or nonactivated form into contact with the product to be tested,
adding, under suitable conditions, an aptamer of claim 7, before, at the same time as or after the product to be tested,
evaluating the competitive binding between the aptamer and the product to be tested.

35. A method for screening products which interact with an RPTK receptor or targets which form a complex with said RPTK in an activated or nonactivated form, which method comprises:
bringing cells expressing RPTKs in an activated or nonactivated form into contact with the substance to be tested,
adding, under suitable conditions, an aptamer of claim 7, before, at the same time as or after the substance to be tested,
evaluating the competitive binding between the aptamer and the molecule to be tested.

36. The method as claimed in claim 35, wherein after identification of the substances which bind competitively with the aptamer to the cells exhibiting RPTKs, the effect of these substances on the biological activity of said cells can be evaluated in order to find substances which inhibit or activate biological activities of the cells exhibiting RPTKs.

37. The method of claim 1 wherein the starting nucleic acid combinatorial library contains nucleic acids comprising random sequences characterized by respectively at their 5′ and 3′ ends having fixed sequences for PCR amplification.

38. The aptamer as claimed in claim 9 wherein said aptamer recognises the Ret receptor activated by mutation at a cysteine located in the extracellular domain.

39. The aptamer as claimed in claim 9 wherein said aptamer recognises the Ret receptor activated by mutation at a cysteine located at codons 609, 611, 618, 620 or 634.

40. The aptamer as claimed in claim 13 wherein the riboses of the purines bear a hydroxyl function on the carbon in the 2′-position, while the riboses of the pyrimidines bear a fluorine atom on the carbon in the 2′-position.

41. The aptamer as claimed in claim 14 wherein said aptamer has formula II below:
5′R₄X₆X₅X₄X₃GGAAUAGX₂X₁R₃X′₁X′₂CGUAUACX′₃X′₄X′₅X′₆R₅3′ (SEQ ID NO: 34) (II), wherein:
the riboses of the purines bear an OH group in the 2′-position and the riboses of the pyrimidines bear a fluorine atom in the 2′-position;
R₃ is present or absent and represents an apical bulge comprising:
a linear or branched carbon chain selected from the group consisting of C₆-C₃₀ alkyl groups and C₆-C₃₀ aryl groups,
a polymer selected from the group consisting of PEG and PEI,
functional groups selected from the group consisting of biotin, streptavidin and peroxidase,
other molecules of interest selected from the group consisting of active ingredients, labeling tags and chelating agents for radioisotopes,
a natural or modified nucleotide sequence;
X₁, X′₁, X₂, X′₂, X₃, X′₃, X₄, X′₄, X₅, X′₅, X₆ and X′₆ represent Py or Pu with
X₁-X′₁ corresponding to C-G, A-U, G-C or U-A
X₂-X′₂ corresponding to C-G, A-U, G-C or U-A
X₃-X′₃ corresponding to C-G, A-U, G-C or U-A
X₄-X′₄ corresponding to C-G, A-U, G-C or U-A
X₅-X′₅ corresponding to C-G, A-U, G-C or U-A
X₆-X′₆ corresponding to C-G, A-U, G-C or U-A
N corresponding to G or C or A or U,
Pu corresponding to G or A, in which the riboses bear an OH group in the 2′-position,
Py corresponds to U or C, in which the riboses bear a fluorine atom in the 2′-position, and
R₄ and R₅ are present or absent and represent:
a natural or modified nucleotide sequence, comprising between 1 and several thousand nucleotides, wherein a part of said nucleotide sequence is selected from the group consisting of the following sequences:
R₄:
5′-R₁-Z₁-3′, with Z₁ = G:
(SEQ ID NO:18)
5′ GGGAGACAAGAAUAAACGCUCAAG 3′,
5′-R₁-Z₁-3′, with Z₁ = GCGGUAU (SEQ ID NO:26):
(SEQ ID NO:19)
5′ GGGAGACAAGAAUAAACGCUCAAGCGGUAU,
and
R₅:
5′-Z₂-R₂-3′, with Z₂ = CAAUCCAGGGCAACG (SEQ ID
NO:27):
(SEQ ID NO:20)
5′CAAUCCAGGGCAACGAACGACAGGAGGCUCACAACAGGA 3′
5′-Z₂-R₂-3′, with Z₂ = ACCGCAGCG (SEQ ID NO:28):
(SEQ ID NO:21)
5′ ACCGCAGCGAACGACAGGAGGCUCACAACAGGA 3′,
(SEQ ID NO:18)
5′ GGGAGACAAGAAUAAACGCUCAAG 3′
(SEQ ID NO:19)
5′ GGGAGACAAGAAUAAACGCUCAAGCGGUAU,
for R₄
and
(SEQ ID NO:20)
5′ CAAUCCAGGGCAACGAACGACAGGAGGCUCACAACAGGA 3′
and
(SEQ ID NO:21)
5′ ACCGCAGCGAACGACAGGAGGCUCACAACAGGA 3′
for R₅;
a linear or branched carbon chain selected from the group consisting of C₆-C₃₀ alkyl groups, and C₆-C₃₀ aryl groups;
a polymer selected from the group consisting of PEG and PEI;
functional groups selected from the group consisting of biotin, streptavidin and peroxidase;
other molecules of interest selected from the group consisting of active ingredients, labeling tags and chelating agents for radioisotopes.

42. A pharmaceutical composition, comprising:
an aptamer as claimed in claim 7, which has both an ability to bind to Ret receptor mutated at one of the cysteines located in the extracellular domain (codons 609, 611, 618, 620 and 634), and an inhibitory action with respect to this mutated receptor,
another anticancer molecule, and
at least one pharmaceutically acceptable vehicle.

43. The aptamer as claimed in claim 16, wherein
R₃ represents bulges selected from the group consisting of (1) to (4):

| | |
|---|---|
| loop (1): 5′ UGGAAGGA 3′ | (SEQ ID NO:29) |
| loop (2): 5′ CUUUUUU 3′ | (SEQ ID NO:30) |
| loop (3): 5′ GNPuA 3′ | |
| loop (4): 5′ UNCG 3′, | |

in which the riboses of the purines bear a hydroxyl function on the carbon in the 2′ position, while the riboses of the pyrimidines bear a fluorine atom on the carbon in the 2′-position.

44. The aptamer as claimed in claim 41, wherein
R₃ represents bulges selected from the group consisting of (1) to (4):

| | |
|---|---|
| loop (1): 5′ UGGAAGGA 3′ | (SEQ ID NO:29) |
| loop (2): 5′ CUUUUUU 3′ | (SEQ ID NO:30) |
| loop (3): 5′ GNPuA 3′ | |
| loop (4): 5′ UNCG 3′, | |

in which the riboses of the purines bear a hydroxyl function on the carbon in the 2′ position, while the riboses of the pyrimidines bear a fluorine atom on the carbon in the 2′-position.

45. The method of claim 1 wherein the starting nucleic acid combinatorial library contains nucleic acids comprising the sequences SEQ ID NO: 1, SEQ ID NO:2 or a fragment of at least 8 nucleotides of these sequences.
